(19) United States
(12) Patent Application Publication
(10) Pub. No.: US 2022/0211657 A1
(43) Pub. Date: Jul. 7, 2022

(54) METHODS OF TREATING GASTROINTESTINAL CANCERS AND TUMORS THEREOF USING COMBINATION THERAPY

(71) Applicant: GEORGETOWN UNIVERSITY, Washington, DC (US)

(72) Inventors: Michael J. PISHVAIAN, Rockville, MD (US); Jonathan R. BRODY, Philadelphia, PA (US); John L. MARSHALL, Arlington, VA (US)

(21) Appl. No.: 17/611,283

(22) PCT Filed: May 13, 2020
(86) PCT No.: PCT/US20/32690

§ 371 Date: Nov. 15, 2021

Related U.S. Application Data
(60) Provisional application No. 62/847,861, filed on May 14, 2019.

(51) Int. Cl. A61K 31/282; A61K 45/06; A61P 35/04

(52) U.S. Cl. CPC A61K 31/282; A61P 35/04; A61K 45/06

(57) ABSTRACT

Aspects of the technology described herein are directed to a method of treating gastrointestinal cancer in a subject. This method involves selecting a subject, where the subject (i) has been diagnosed with a gastrointestinal cancer and (ii) has (a) a pathogenic mutation in one or more homologous recombination-DNA damage repair (HR-DDR) pathway genes and/or (b) a family history suggestive of a breast or ovarian cancer syndrome; and administering to the subject an effective amount of a Poly(ADP ribose) polymerase (PARP) inhibitor, in combination with oxaliplatin and an antimetabolite. Methods of treating a gastrointestinal tumor in a subject and of increasing sensitivity of gastrointestinal tumor cells to oxaliplatin are also disclosed.

This application claims the priority benefit of U.S. Provisional Patent Application Ser. No. 62/847,861, filed May 14, 2019, which is hereby incorporated by reference in its entirety.

### FIELD

Aspects of the technology described herein relate to methods of treating gastrointestinal cancers and tumors, as well as methods of increasing sensitivity of gastrointestinal tumor or gastrointestinal cancer cells to a platinum-based chemotherapy.

### BACKGROUND

Pancreatic cancer is one of the deadliest malignancies which, with a 5 year overall survival of only 9%, is poised to become the second leading cause of cancer related death in the United States by 2020 (Rahib et al., “Projecting Cancer Incidence and Deaths to 2030: The Unexpected Burden of Thyroid, Liver, and Pancreas Cancers in the United States,” Cancer Res. 74:2913-2921 (2014)). Treatment for metastatic pancreatic cancer has improved, but the median overall survival remains less than 1 year (Conroy et al., “FOLFIRINOX Versus Gemcitabine for Metastatic Pancreatic Cancer,” N. Engl. J. Med. 364:1817-1825 (2011); Von Hoff et al., “Increased Survival in Pancreatic Cancer with Nab-Paclitaxel Plus Gemcitabine,” N. Engl. J. Med. 369:1691-1703 (2013)).

For a subgroup of up to 17% of patients with pancreatic cancer whose tumors harbor underlying defects in the homologous recombination-DNA damage repair (HR-DDR) pathway (Pishvaian et al., “Molecular Profiling of Patients with Pancreatic Cancer: Initial Results from the Know Your Tumor Initiative,” Clin. Cancer Res. 24:5018-5027 (2018). Aguirre et al., “Real-Time Genomic Characterization of Advanced Pancreatic Cancer to Enable Precision Medicine,” Cancer Discov. 8:1096-1111 (2018); Lowery et al., “Real-Time Genomic Profiling of Pancreatic Ductal Adenocarcinoma: Potential Actionability and Correlation with Clinical Phenotype,” Clin. Cancer Res. 23:6094-6100 (2017); Bailey et al., “Genomic Analyses Identify Molecular Subtypes of Pancreatic Cancer,” Nature 531:47-52 (2016)), such as BRCA1/2 and PALB2 mutations, treatment with platinum-based chemotherapy and poly(ADP-ribose) polymerase (PARP) inhibitors have been attempted (Domchek et al., “Efficacy and Safety of Olaparib Monotherapy in Germlinc BRCA1/2 Mutation Carriers with Advanced Ovarian Cancer and Three or More Lines of Prior Therapy,” Gynecol. Oncol. 140:199-203 (2016); Golan et al., “Overall Survival and Clinical Characteristics of BRCA Mutation Carriers With Stage I/II Pancreatic Cancer,” Br. J. Cancer 116:697-702 (2017); Lowery et al., “An Emerging Entity: Pancreatic Adenocarcinoma Associated with a Known BRCA Mutation: Clinical Descriptors, Treatment Implications, and Future Directions,” Oncologist 16:1397-1402 (2011); O'Reilly et al., “Phase 1 Trial Evaluating Cisplatin, Gemcitabine, and Veliparib in 2 Patient Cohorts: Germline BRCA Mutation Carriers and Wild-Type BRCA Pancreatic Ductal Adenocarcinoma,” Cancer (2018); O'Reilly et al., “Phase IB Trial of Cisplatin (C), Gemcitabine (G), and Veliparib (V) in Patients With Known or Potential BRCA or PALB2-Mutated Pancreas Adenocarcinoma (PC),” JCO 32:5s, (suppl; abstr 4023) (2014); Shroff et al., “Rucaparib Monotherapy in Patients with Pancreatic Cancer and a Known Deleterious BRCA Mutation,” JCO Precis. Oncol. (2018)).

PARP is a nuclear enzyme that plays a critical role in DNA damage repair (Lord et al., “PARP Inhibitors: Synthetic Lethality in the Clinic,” Science 355:1152-1158 (2017); del Rivero et al., “PARP Inhibitors: The Cornerstone of DNA Repair-Targeted Therapies,” Oncology (Williston Park) 31:265-273 (2017); O'Connor M J, “Targeting the DNA Damage Response in Cancer,” Mol. Cell 60:547-560 (2015): Golan et al., “DNA Repair Dysfunction in Pancreatic Cancer: A Clinically Relevant Subtype for Drug Development,” J. Natl. Compr. Canc. Netw. 15:1063-1069 (2017); Lord et al., “Targeted Therapy for Cancer Using PARP Inhibitors,” Curr. Opin. Pharmacol. 8:363-369 (2008)). Inactive PARP is autoactivated upon binding to damaged DNA and subsequently poly(ADP-ribosyl)ates many nuclear target proteins, including those that facilitate the repair of both single-stranded and double-stranded DNA breaks (Ratnam et al., “Current Development of Clinical inhibitors of Poly(ADP-ribose) Polymerase in Oncology,” Clin. Cancer Res. 13:1383-1388 (2007). Thus, PARP inhibition results in less efficient DNA repair following a cytotoxic insult, and PARP inhibitors may act as sensitizing agents for a variety of DNA-damaging chemotherapeutic agents (Steffen et al., “Targeting PARP-1 Allosteric Regulation Offers Therapeutic Potential Against Cancer,” Cancer Res. 74:31-37 (2014)).

PARP inhibitors can have multiple effects in mediating DNA damage, leading to cancer cell death. PARP inhibitors inhibit single strand repair. However, some PARP inhibitors trap the PARP enzyme at sites of DNA damage, resulting in replication fork arrest, leading ultimately to mitotic catastrophe and apoptotic cell death. Several PARP inhibitors such as olaparib, niraparib, rucaparib, and talozaparib can achieve PARP trapping and replication fork arrest, and thus are active as single agents. However, many such PARP trapping inhibitors are too toxic to use in combination with DNA damaging chemotherapies (Chen et al., “A Phase I Study of Olaparib and Irinotecan in Patients with Colorectal Cancer: Canadian Cancer Trials Group IND 187,” Invest. New Drugs 34(4):450-457 (2016); Samol et al., “Safety and Tolerability of the Poly(ADP-ribose) Polymerase (PARP) Inhibitor, Olaparib (AZD2281) in Combination with Topotecan for the Treatment of Patients with Advanced Solid Tumors: A Phase I Study,” Invest. New Drugs 30(4):1496-1500 (2012); Balmaña et al., “Phase I Trial of Olaparib in Combination with Cisplatin for the Treatment of Patients with Advanced Breast, Ovarian and other Solid Tumors,” Ann. Oncol. 25(8):1656-1663 (2014); Rajan et al., “A Phase I Combination Study of Olaparib with Cisplatin and Gemcitabine in Adults with Solid Tumors,” Clin. Cancer Res. 18(8):2344-2351 (2012); Dhawan et al., “Differential Toxicity in Patients with and without DNA Repair Mutations: Phase I Study of Carboplatin and Talazoparib in Advanced Solid Tumors,” Clin. Cancer Res. 23(21):6400-6410 (2017)). Thus, there remains a need for effective pancreatic cancer therapies with improved treatment outcomes.

The present invention is directed to overcoming these and other deficiencies in the art.

### SUMMARY

One aspect of the technology described herein relates to a method of treating gastrointestinal cancer in a subject. This method comprises: selecting a subject, wherein the subject (i) has been diagnosed with gastrointestinal cancer and (ii) has (a) a pathogenic mutation in one or more homologous recombination-DNA damage repair (HR-DDR) pathway genes and/or (b) a family history suggestive of a breast or ovarian cancer syndrome; and administering to the subject an effective amount of a Poly(ADP ribose) polymerase (PARP) inhibitor, in combination with oxaliplatin and an antimetabolite.

Another aspect of the technology described herein relates to a method of treating a gastrointestinal tumor in a subject. This methods comprises: selecting a gastrointestinal tumor of a subject, wherein the tumor has a pathogenic mutation in one or more HR-DDR pathway genes and/or the subject has a family history suggestive of a breast or ovarian cancer syndrome; and administering to the tumor an effective amount of a PARP inhibitor, in combination with oxaliplatin and an antimetabolite.

A further aspect of the technology described herein relates to a method of increasing sensitivity of a gastrointestinal tumor cell or gastrointestinal cancer cell to treatment with oxaliplatin. This method comprises: selecting a gastrointestinal tumor cell or gastrointestinal cancer cell, wherein the cell comprises a pathogenic mutation in one or more HR-DDR pathway genes; and administering to the cell a PARP inhibitor in an amount effective to increase sensitivity of the cell to treatment with oxaliplatin and an antimetabolite.

As described herein, the present disclosure demonstrates, inter alia, that a combination therapy that includes a PARP inhibitor, oxaliplatin, and an antimetabolite is surprisingly effective in treating pancreatic cancer in patients that have an HR-DDR pathway mutation and/or a family history suggestive of a breast or ovarian cancer syndrome. It is expected that the combination therapy is also effective in treating other gastrointestinal cancers in patients with such a mutation and/or family history.

### BRIEF DESCRIPTION OF THE DRAWINGS

FIG. 1 is a flowchart of the treatment cohorts. Of the 75 patients consented, 64 initiated study treatment. Two of the six patients in the Phase I cohort with the 5-fluorouracil (5FU) bolus came off due to toxicity before response evaluation. In the Phase I portion without the 5FU bolus, 1 patient withdrew consent, 1 patient came off due to a perforated gall bladder, and 1 patient could not swallow the pills; 1 patient was also lost to follow up before response assessment. In the Phase II cohorts, all patients were evaluable for toxicity and response.

FIG. 2 is a waterfall plot of patient responses.

FIG. 3 is a swimmers plot of patient progression-free survival and overall survival.

FIGS. 4A-4B relate to the pharmacokinetic analysis of 14 subjects in 5 veliparib dosing cohorts. FIG. 4A is a table showing the number of subjects in each cohort. FIG. 4B is a table showing the pharmacokinetic values on Days 1, 3, and 7.

### DETAILED DESCRIPTION

In this specification and the appended claims, the singular forms “a”, “an”, and “the” include plural references unless the context clearly dictates otherwise.

The terms “comprising”. “comprises” and “comprised of” as used herein are synonymous with “including”, “includes” or “containing”, “contains”, and are inclusive or open-ended and do not exclude additional, non-recited members, elements, or method steps.

The terms “comprising”, “comprises”, and “comprised of” also encompass the term “consisting of”.

The recitation of numerical ranges by endpoints includes all numbers and fractions subsumed within the respective ranges, as well as the recited endpoints.

One aspect of the technology described herein relates to a method of treating gastrointestinal cancer in a subject. This method involves selecting a subject who has been diagnosed with a gastrointestinal cancer and has a pathogenic mutation in one or more homologous recombination-DNA damage repair (HR-DDR) pathway genes, a family history suggestive of a breast or ovarian cancer syndrome, or both; and administering to the subject an effective amount of a Poly(ADP ribose) polymerase (PARP) inhibitor, in combination with oxaliplatin and an antimetabolite. Another aspect of the technology described herein relates to a method of treating a gastrointestinal tumor in a subject. This methods involves selecting a gastrointestinal tumor of a subject, wherein the tumor has a pathogenic mutation in one or more HR-DDR pathway genes and/or the subject has a family history suggestive of a breast or ovarian cancer syndrome. This method further involves administering to the tumor an effective amount of a PARP inhibitor, in combination with oxaliplatin and an antimetabolite.

In some embodiments of the methods described herein, the subject, the tumor, or the cell is selected based on the subject, tumor, or cell having a pathogenic mutation in one or more HR-DDR pathway genes. HD-DDR refers to the process of repairing DNA damage using a homologous nucleic acid. In a normal cell, HD-DDR typically involves a series of steps such as recognition of a DNA break, stabilization of the break, resection, stabilization of single stranded DNA, formation of a DNA crossover intermediate, resolution of the crossover intermediate, and ligation. Pathogenic mutations according to the methods described herein include those that are likely pathogenic (at least 0.95 probability) and definitely pathogenic (>0.99 probability) (e.g., Plon et al., Human Mutation 29:1282-91 (2008), which is hereby incorporated by reference in its entirety).

In some embodiments, the one or more HR-DDR pathway genes is selected from the group consisting of ARID1A, ATM, ATRX, MRE11A, NBN, PTFN, RAD50/51/51B, BARD1, BLM, BRCA1, BRCA2, BRIP1, FANCA/C/D2/E/F/G/L, PALB2, WRN, CHEK1, CHEK2, BAP1, FAM175A, SLX4, MLL2, and XRCC. Pathogenic mutations can be identified using standard techniques. For example, commercial and/or research testing laboratories can be used to screen for mutations known to be pathogenic.

In some embodiments, the one or more HR-DDR pathway genes is BRCA1, BRCA2, PALB2, or any combination thereof.

BRCA1 is a gene that encodes a polypeptide with a zinc finger domain and a BRCT domain, which is involved in DNA damage repair. BRCA1 binds to DNA and interacts directly with RAD51. BRCA1 gene sequences for various species are known in the art and include, e.g., human BRCA1 (NCBI Gene ID: 672): mouse BRCA1 (NCBI Gene ID: 12189); Norway rat BRCA1 (NCBI Gene ID: 497672): dog BRCA1 (NCBI Gene ID: 403437); cattle BRCA1 (NCBI Gene ID: 353120); rhesus monkey BRCA1 (NCBI Gene ID: 712634); and pig BRCA1 (NCBI Gene ID: 100049662).

BRCA2 is a gene that encodes a tumor suppressor that normally functions by binding single-stranded DNA at DNA damage sites and interacting with RAD51 to promote strand invasion. BRCA2 gene sequences for various species are known in the art and include, e.g., human BRCA2 (NCBI Gene ID: 675); mouse BRCA2 (NCBI Gene ID: 12190); Norway rat BRCA2 (NCBI Gene ID: 360254); dog BRCA2 (NCBI Gene ID: 474180): cattle BRCA2 (NCBI Gene ID: 507069); rhesus monkey BRCA2 (NCBI Gene ID: 721981); and pig BRCA2 (NCBI Gene ID: 100624979).

Examples of pathogenic BRCA1 and BRCA2 mutations include, without limitation, those listed in the University of Utah Department of Pathology and ARUP Laboratories BRCA mutation database (http://arup.utah.edu/database/BRCA/Variants/BRCA1.php: http://arup.utah.edu/database/BRCA/Variants/BRCA2.php, each of which is hereby incorporate by reference in its entirety).

PALB2 is a gene that encodes a DNA-binding protein (Partner And Localizer of BRCA2) that binds to single-strand DNA and facilitates accumulation of BRCA2 at the site of DNA damage. PALB2 also interacts with RAD51 to promote strand invasion. PALB2 gene sequences for various species are known in the art and include, e.g., human PALB2 (NCBI Gene ID: 79728); mouse PALB2 (NCBI Gene ID: 233826); Norway rat PALB2 (NCBI Gene ID: 293452); dog PALB2 (NCBI Gene ID: 608527); cattle PALB2 (NCBI Gene ID: 507620); rhesus monkey PALB2 (NCBI Gene ID: 700843): and pig PALB2 (NCBI Gene ID: 100523630). Examples of pathogenic PALB2 mutations include those listed in Kim et al., “Frequency of Pathogenic Germline Mutation in CHEK2, PALB2, MRE11, and RAD50 in Patients at High Risk for Hereditary Breast Cancer,” Breast Cancer Res. Treat. 161(1):95-102; Girard et al., “Familial Breast Cancer and DNA Repair Genes: Insights into Known and Novel Susceptibility Genes from the GENESIS Study, and Implications for Multigene Panel Testing,” Int. J. Cancer 144(8):1962-1974 (2019), Zhan et al., “Germline Variants and Risk for Pancreatic Cancer: A Systematic Review and Emerging Concepts,” Pancreas 47(8):924-936; Reid et al., “Biallelic Mutations in PALB2 cause Fanconi Anemia Subtype FA-N and Predispose to Childhood Cancer” Nat. Genet. 39(2):162-164 (2007); and Janatova et al., “The PALB2 Gene is a Strong Candidate for Clinical Testing in BRCA1- and BRCA2-Negative Hereditary Breast Cancer,” Cancer Epidemiol. Biomarkers Prev. 22(12):2323-2332 (2013), each of which is hereby incorporated by reference in its entirety).

In the context of the methods described herein, a pathogenic mutation may be a germline mutation or a somatic mutation. As used herein, the term “germline mutation” refers to a mutation that is transmitted from one organismic generation to the next. As used herein, the term “somatic mutation” refers to a mutation that strikes the genome of a cell outside of the germline; such a mutation cannot, by definition, be transmitted to the next organismic generation. In some embodiments, the pathogenic mutation is a germline mutation in BRCA1, BRCA2, PALB2, or any combination thereof. Germline BRCA1 and BRCA2 mutations are found in approximately 5% to 10% of familial pancreatic ductal adenocarcinoma (“PDAC”) and approximately 3% of apparently sporadic PDAC (Blair et al., “BRCA1/BRCA2 Germline Mutation Carriers and Sporadic Pancreatic Ductal Adenocarcinoma,” J. Am. Coll. Surg. 226(4):630-637 (2018), which is hereby incorporated by reference in its entirety). PALB2 binds to and colocalizes with BRCA2 in DNA repair. Germline mutations in PALB2 have been identified in approximately 3-4% of familial pancreatic cancer cases (Hofstatter et al., “PALB2 Mutations in Familial Breast and Pancreatic Cancer,” Fam. Cancer 10(2):10.1007/s10689-011-92426.1 (2011), which is hereby incorporated by reference in its entirety).

In some embodiments, the subject or tumor of a subject is selected based on the subject having a family history suggestive of a breast or ovarian cancer syndrome. As will be apparent to the skilled artisan, a family history suggestive of a breast or ovarian cancer syndrome can be determined using, for example, established clinical guidelines, such as those set forth by the National Comprehensive Cancer Network or similar organization (e.g., NCCN Clinical Practice Guidelines in Oncology, “Genetic/Familial High-Risk Assessment: Breast and Ovarian, Version 3.2019,” J. Natl. Compr. Canc. Netw. (2019), which is hereby incorporated by reference in its entirety). In some embodiments, the subject is one who meets one or more of the following criteria.
A. A personal history of breast cancer and one or more of the following: (1) diagnosed ≤45 years old; (2) diagnosed at any age with one or more 1^{st}, 2^{nd}, or 3^{rd} degree relatives with breast cancer ≤50 years old and/or one or more 1^{st}, 2^{nd}, or 3^{rd} degree relatives with epithelial ovarian cancer at any age; (3) two primary breast cancers with the first diagnosed at ≤50 years old; (4) diagnosed at ≤60 years old with a triple negative breast cancer; (5) diagnosed at any age with two or more 1^{st}, 2^{nd}, or 3^{rd} degree relatives with breast cancer at any age; (6) diagnosed at any age with two or more 1^{st}, 2^{nd}, or 3^{rd} degree relatives with a pancreatic cancer or aggressive prostate cancer (Gleason score 7) at any age; (7) 1^{st}, 2^{nd}, or 3^{rd} degree male relatives with breast cancer; (8) Ashkenazi Jewish descent.
B. A personal history of epithelial ovarian cancer.
C. A personal history of male breast cancer.
D. A personal history of pancreatic cancer and two or more 1^{st}, 2^{nd}, or 3^{rd} degree relatives with breast, epithelial ovarian, pancreatic, or aggressive prostate cancer (Gleason score ≥7) at any age.

Various additional selection criteria can also be used to select suitable subjects/tumors. For example, the subject or tumor of a subject may be selected on the basis of the subject's organ function and/or bone marrow function. Organ function can be measured using methods well known in the art to quantify, e.g., serum creatine levels (kidney function), bilirubin levels (liver function), and ALT/AST levels (liver levels). Bone marrow function can be measured using methods well known in the art to quantify, e.g., hemoglobin levels, absolute neutrophil count, and platelet counts. In some embodiments, the subject has adequate organ and bone marrow function when selected for treatment. In some embodiments, the subject has serum creatine levels <2 mg/dL, bilirubin levels <3× upper limit of normal (ULN), ALT/AST levels <5×ULN, hemoglobin ≥9.5 g/dL, absolute neutrophil count ≥1.5×10⁹/L, and/or a platelet count ≥75×10⁹/L. In some embodiments, the subject has serum creatine levels <1.5 mg/dL, bilirubin levels ≤2.5×ULN, ALT/AST levels ≤3×ULN, hemoglobin ≥9.5 g/dL, absolute neutrophil count ≥1.5×10⁹/L, and/or a platelet count ≥75×10⁹/L.

In some embodiments, additional selection criteria relate to whether the subject has received prior treatment with a platinum based chemotherapy. In the context of the present application, a “platinum based chemotherapy” means any treatment that includes at least a platinum-based compound (i.e., any compound containing a platinum atom capable of binding and cross-linking DNA, inducing the activation of the DNA repair, and ultimately triggering apoptosis). Platinum based compounds include, without limitation, carboplatin, cisplatin, oxaliplatin, iproplatin, nedaplatin, triplatin tetranitrate, tetraplatin, satraplatin, and the like. Various platinum based chemotherapy regimens are well known in the art and include, e.g., the FOLFIRINOX regimen (folinic acid, fluorouracil, irinotecan, and oxaliplatin), the FOLFOX regimen (folinic acid, fluorouracil, and oxaliplatin), and the CAPEOX regimen (capecitabine plus oxaliplatin) (Sobrero et al., “FOLFOX or CAPOX in Stage II to III Colon Cancer: Efficacy Results of the Italian Three or Six Colon Adjuvant Trial,” J. Clin. Oncol. 36(15):1478-1485 (2018), which is hereby incorporated by reference in its entirety). In some embodiments of the methods described herein, the selected subject has received systemic treatment with a platinum based chemotherapy, for any disorder (e.g., for the gastrointestinal cancer to be treated according to the methods described herein, for another gastrointestinal cancer, for a non-gastrointestinal cancer, for a non-cancer disorder), at any time prior to selection. In some embodiments, the selected subject received the prior systemic treatment within 3 months (e.g., within 2 months, 1 month, 4 weeks, 3 weeks, 2 weeks, 1 week) prior to selection. In some embodiments, the disorder did not progress in the subject following the prior systemic treatment. In some embodiments, the disorder did progress in the subject following the prior systemic treatment. In some embodiments, the prior systemic treatment was within 3 months (e.g., within 2 months, 1 month, 4 weeks, 3 weeks, 2 weeks, 1 week) prior to selection and the disorder did not progress following the prior systemic treatment. In some embodiments, the prior systemic treatment was at any time prior to selection and the disorder did progress following the prior systemic treatment. In some other embodiments, the subject has not received systemic treatment with a platinum based chemotherapy, for any disorder, at any time prior to selection. In some other embodiments, the subject has not received systemic treatment with a platinum based chemotherapy for any gastrointestinal cancer at any time prior to selection. In some other embodiments, the subject has not received systemic treatment with a platinum based chemotherapy for the gastrointestinal cancer to be treated according to the methods described herein at any time prior to selection.

Suitable subjects in accordance with the methods described herein include, without limitation, mammals. In some embodiments, the subject is selected from the group consisting of primates (e.g., humans, monkeys), equines (e.g., horses), bovines (e.g., cattle), porcines (e.g., pigs), ovines (e.g., sheep), caprines (e.g., goats), camelids (e.g., llamas, alpacas, camels), rodents (e.g., mice, rats, guinea pigs, hamsters), canines (e.g., dogs), felines (e.g., cats), leporids (e.g., rabbits). In some embodiments, the selected subject is an agricultural animal, a domestic animal, or a laboratory animal. In some embodiments, the subject is a human subject.

As noted above, the subject is one who has been diagnosed with a gastrointestinal cancer, the tumor is a gastrointestinal tumor, or the cell is a gastrointestinal cancer cell.

As used herein, the terms “cancer” and “cancerous” refer to or describe the physiological condition in which a population of cells are characterized by abnormal, unrestrained growth with the potential to cause detrimental local mass effects, or to spread to other parts of the body. Examples of cancer include, but are not limited to, carcinoma, sarcoma, melanoma, leukemia, lymphoma, and combinations thereof (mixed-type cancer). A “carcinoma” is a cancer originating from epithelial cells of the skin or the lining of the internal organs. A “sarcoma” is a tumor derived from mesenchymal cells, usually those constituting various connective tissue cell types, including fibroblasts, osteoblasts, endothelial cell precursors, and chondrocytes. A “melanoma” is a tumor arising from melanocytes, the pigmented cells of the skin and iris. A “leukemia” is a malignancy of any of a variety of hematopoietic stem cell types, including the lineages leading to lymphocytes and granulocytes, in which the tumor cells are nonpigmented and dispersed throughout the circulation. A “lymphoma” is a solid tumor of the lymphoid cells. More particular examples of such cancers include, e.g., acinar cell carcinoma, adenocarcinoma (ductal adenocarcinoma), adenosquamous carcinoma, anaplastic carcinoma, cystadenocarcinoma, duct-cell carcinoma (ductal adrenocarcinoma), giant-cell carcinoma (osteoclastoid type), mixed-cell carcinoma, mucinous (colloid) carcinoma, mucinous cystadenocarcinoma, papillary adenocarcinoma, pleomorphic giant-cell carcinoma, serous cystadenocarcinoma, and small-cell (oat-cell) carcinoma.

As used herein, “gastrointestinal cancer” refers to a condition characterized by cancerous cells that originate in the gastrointestinal tract, an accessory organ of digestion, or the peritoneum. The abnormal cells often are referred to as “neoplastic cells,” which as used herein refers to transformed cells that can form a solid tumor. The term “gastrointestinal tumor” as used herein refers to an abnormal mass or population of cells (i.e., two or more cells) of the gastrointestinal tract, an accessory organ of digestion, or the peritoneum that results from excessive or abnormal cell division. The terms “cancer cell” and “tumor cell” refer to one or more cells derived from a tumor or cancerous lesion.

As used herein, the “gastrointestinal tract” refers to the entire alimentary canal, from the oral cavity to the rectum. The gastrointestinal tract includes the oral cavity (mouth or buccal cavity), pharynx (throat), esophagus, stomach, small intestine, and large intestine (cecum, colon, rectum, anus). As used herein, the an “accessory organ of digestion” is an organ that supports the functions of the gastrointestinal tract including, e.g., the salivary glands, liver, pancreas, and gallbladder, which secrete various hormones and/or digestive enzymes. For example, salivary glands secrete digestive enzymes and saliva: the liver produces bile, which is stored, concentrated, and released by the gallbladder; and the pancreas is a compound gland that discharges digestive enzymes into the gut and secretes the hormones insulin and glucagon into the bloodstream.

The gastrointestinal cancer/tumor may be an oral cavity cancer/tumor, pharyngeal cancer/tumor, esophageal cancer/tumor, stomach (i.e., gastric) cancer/tumor, small intestinal cancer/tumor, cecal cancer/tumor, colon cancer/tumor (including colorectal cancer/tumor), rectal cancer/tumor, anal cancer/tumor, salivary gland cancer/tumor, liver cancer/tumor, pancreatic cancer/tumor, biliary cancer/tumor (bile duct cancer/tumor), gall bladder cancer/tumor, or peritoneal cancer/tumor.

The oral cavity includes the lips, the inside lining of the lips and cheeks, the teeth, the gums, the front two-thirds of the tongue, the floor of the mouth below the tongue, and the bony roof of the mouth (hard palate). The oropharynx is the part of the throat just behind the mouth. It starts where the oral cavity stops. It includes the base of the tongue (the back third of the tongue), the soft palate (the back part of the roof of the mouth), the tonsils, and the side and back walls of the throat. Exemplary oral cavity, pharyngeal, and/or oropharyngeal cancers/tumors include, but are not limited to, squamous cell carcinomas (carcinoma in situ, verrucous carcinoma).

The esophagus is a hollow, muscular tube that connects the throat to the stomach. Exemplary esophageal cancers/tumors include, but are not limited to, adenocarcinoma, squamous cell carcinoma, small cell carcinoma, lymphoma, melanomas, and sarcoma.

The stomach receives food from the esophagus and secretes digestive enzymes. Exemplary gastric cancers/tumors include, but are not limited to, adenocarcinoma (distal stomach cancer, proximal stomach cancer, diffuse stomach cancer), gastrointestinal stromal tumors, carcinoid tumors, lymphoma, squamous cell carcinoma, small cell carcinoma, leiomyosarcoma, signet ring cell carcinoma, gastric lymphoma (MALT lymphoma), and linitis plastica.

The small intestine receives partially digested food from the stomach, continues digesting food, and absorbs nutrients. Exemplary small intestinal cancers/tumors include, but are not limited to, adenocarcinoma, carcinoid tumors, lymphomas, and sarcomas (gastrointestinal stromal tumors).

The large intestine comprises the cecum, colon, rectum, and anal canal. The cecum is the portion of the large intestine that connects the ileum of the small intestine to the colon. Exemplary cecal cancers/tumors include, but are not limited to, adenocarcinoma, squamous cell carcinoma, and sarcoma (leiomyosarcoma). The colon receives almost completely digested food from the cecum, absorbs water and nutrients, and passes waste to the rectum. Exemplary colon cancers/tumors include, but are not limited to, adenocarcinoma, carcinoid tumors, gastrointestinal stromal tumors, lymphomas, and sarcomas. The rectum receives waste from the colon and stores it until it passes out of the body through the anus. Exemplary rectal cancers/tumors include, but are not limited to, adenocarcinoma, carcinoid tumors, gastrointestinal stromal tumors, lymphomas, and sarcomas. Colorectal cancers/tumors involve both the colon and the rectum. The anus is the opening at the lower end of the rectum through which waste is passed from the body. Exemplary anal cancers/tumors include, but are not limited to, carcinoma in situ (Bowen disease), squamous cell carcinomas (e.g., cloacogenic carcinoma), adenocarcinomas, basal cell carcinomas, melanomas, and gastrointestinal stromal tumors.

As used herein, the term “exocrine” refers to a gland that releases a secretion external to or at the surface of an organ by means of a canal or duct. Suitable exocrine glands include, e.g., the salivary gland, liver, and pancreas.

The salivary glands make saliva, which contains enzymes that begin the process of food digestion. Exemplary salivary gland cancers/tumors include, e.g., adenoid cystic carcinoma, mucoepidermoid carcinoma, and polymorphous low-grade adenocarcinoma.

The liver breaks down and stores many of the nutrients absorbed from the intestine, makes clotting factors, delivers bile into the intestines to help absorb nutrients, and breaks down alcohol, drugs, and toxic wastes in the blood. Exemplary liver cancers/tumors include, without limitation, hepatocellular carcinoma (e.g., fibrolamellar hepatocellular carcinoma), intrahepatic cholangiocarcinoma (bile duct cancer), angiosarcoma, hemangiosarcoma, and hepatoblastoma.

As described above, the pancreas is a compound gland that discharges digestive enzymes into the gut (exocrine function) and secretes the hormones insulin and glucagon into the bloodstream (endocrine function). The pancreatic cancer/tumor may be an exocrine cancer/tumor. Exemplary pancreatic cancers/tumors include, but are not limited to, acinar cell carcinoma, adenocarcinoma (ductal adenocarcinoma), adenosquamous carcinoma, anaplastic carcinoma, cystadenocarcinoma, duct-cell carcinoma (ductal adrenocarcinoma), giant-cell carcinoma (osteoclastoid type), a giant cell tumor, intraductal papillary-mucinous neoplasm (IPMN), mixed-cell carcinoma, mucinous (colloid) carcinoma, mucinous cystadenocarcinoma, papillary adenocarcinoma, pleomorphic giant-cell carcinoma, serous cystadenocarcinoma, small-cell (oat-cell) carcinoma, solid tumors, and pseudopapillary tumors.

The bile duct connects the liver, gallbladder, and small intestine. Exemplary biliary cancers/tumors include, but are not limited to, adenocarcinomas, sarcomas, lymphomas, and small cell cancers. Bile duct cancers may also be classified by location as intrahepatic bile duct cancer, perihilar bile duct cancer, and distal bile duct cancer.

The gall bladder is a small, pear-shaped organ that concentrates and stores bile, which is made in the liver. The cystic duct of the gall bladder joins with the common hepatic duct from the liver to form the common bile duct, which joins with the pancreatic duct to empty into the first portion of the small intestine (the duodenum). Gall bladder cancers/tumors include, but are not limited to, adenocarcinomas (papillary adenocarcinoma), adenosquamous carcinomas, squamous cell carcinomas, and carcinosarcomas.

The peritoneum surrounds the organs of the digestive system. Exemplary peritoneal cancers/tumors include, but are not limited to, peritoneal carcinoma, peritoneal mesothelioma, and desmoplastic small round cell tumor.

Malignant tumors are distinguished from benign growths or tumors in that, in addition to uncontrolled cellular proliferation, they can invade surrounding tissues and can metastasize. The term “metastasis” or “metastasize” as used herein refers to a process in which cancer cells travel from one organ or tissue to another non-adjacent organ or tissue. Gastrointestinal tract cancer cells often invade lymph node cells and/or metastasize to the lung and/or bone and spread cancer in these tissues and organs (Riihimäki et al., “Metastatic Spread in Patients with Gastric Cancer,” Oncotarget 7(32):52307-52316 (2016); Riihimäki et al., “Patterns of Metastasis in Colon and Rectal Cancer,” Sci. Rep. 6:29765 (2016), each of which is hereby incorporated by reference in its entirety). In some embodiments of the methods described herein, the gastrointestinal tumor/cancer is a metastatic gastrointestinal tumor/cancer.

In the methods described herein, a combination therapy is administered to the selected subject/tumor. This combination therapy comprises a PARP inhibitor, in combination with oxaliplatin and an antimetabolite.

PARP is a nuclear enzyme that plays a critical role in DNA damage repair. Without wishing to be bound by theory, inhibition of PARP results in less efficient DNA repair following a cytotoxic insult and may sensitize a cancer and/or tumor cell to treatment with DNA-damaging chemotherapeutic agents. Suitable PARP inhibitors for use in the methods described herein include, without limitation, olaparib (AZD 2281), rucaparib (AG 014699), niraparib (MK 4827), talozaparib (BMN 673), and veliparib (ABT-888). In some embodiments, the PARP inhibitor is veliparib (ABT-888).

As used herein, “antimetabolite” refers to a substance that interferes with one or more enzymes or their reactions that are necessary for nucleic acid (DNA and RNA) synthesis. Suitable antimetabolites for use in the methods described herein include, without limitation, 5-fluorouracil (5-FU) and S-1.

In some embodiments, the antimetabolite is 5-FU. 5-flurouracil (5-FU) is a pyrimidine antagonist comprising a pyrimidine base with a fluoride atom at the 5 carbon position on the ring. Uracil is a naturally occurring pyrimidine base used in nucleic acid synthesis, which is converted to thymidine by enzyme action. 5-FU is similar in structure to uracil and is converted to two active metabolites (FdUMP and FUTP) that inhibit the activity of the enzyme thymidylate synthetase. This enzyme normally converts uracil to thymidine by adding a methyl group at the fifth carbon of the pyrimidine ring. 5-FU mimics the natural base and functions to inhibit DNA synthesis. The carbon group cannot be added due to the fluoride atom at the five position and, thus, normal DNA synthesis fails, dUTP and FdUTP are incorporated into DNA so that it cannot function normally. In addition, FUTP is incorporated into RNA leading to faulty translation of the RNA. Thus, the synthesis of multiple forms of RNA (messenger, ribosomal, transfer, and small nuclear RNAs) is blocked. These combined actions on DNA and RNA are cytotoxic to the rapidly dividing cancer cells.

In some embodiments, the antimetabolite is S-1. S-1 consists of three pharmacological agents, at a molar ratio of 1:0.4:1—Tegafur (FT), a prodrug of 5-FU; 5-Chloro-2-4-Dihydroxypyridine (CDHP), which inhibits the activity of Dihydropyrimidine Dehydrogenase (DPD); and Oxonic Acid (Oxo), which reduces gastrointestinal toxicity of 5-FU (Chhetri et al., “Current Development of Anti-Cancer Drug S-1,” J Clin. Diagn. Res. 10(11): XE01-XE05 (2016), which is hereby incorporated by reference in its entirety).

In the methods described herein, therapeutic agents are administered in an effective amount. An effective amount is an amount that, when the therapeutic agents are administered over a particular time interval, results in achievement of one or more therapeutic benchmarks (e.g., slowing or halting of tumor growth, tumor regression, cessation of symptoms, etc.).

The therapeutic agents may be administered to a subject, tumor, or cell one time or multiple times. In those embodiments where the compounds are administered multiple times, they may be administered at a set interval, e.g., daily, every other day, weekly, biweekly, or monthly. Alternatively, they can be administered at an irregular interval, for example on an as-needed basis based on symptoms, patient health, and the like. For example, an effective amount may be administered once a day (q.d.) for one day, at least 2 days, at least 3 days, at least 4 days, at least 5 days, at least 6 days, at least 7 days, at least 10 days, or at least 15 days. Optionally, the status of the cancer or the regression of the tumor is monitored during or after the treatment, for example, by a FES-PET scan. The dosage of the combination administered to the subject can be increased or decreased depending on the status of the cancer or the regression of the tumor detected.

The skilled artisan can readily determine the effective amount, on either an individual subject basis (e.g., the amount of a compound necessary to achieve a particular therapeutic benchmark in the subject being treated) or a population basis (e.g., the amount of a compound necessary to achieve a particular therapeutic benchmark in the average subject from a given population).

Suitable therapeutic benchmarks for treating a gastrointestinal cancer in a subject include, for example, halting disease progression in the subject, inhibiting malignant tumor growth in the subject, inhibiting metastasis of the cancer in the subject, reducing tumor size in the subject, and combinations thereof. Inhibiting according to all methods described herein includes any decrease in growth, metastasis, etc., whether partial or complete.

In some embodiments, halting disease progression in the subject includes increasing the duration of progression-free survival of the subject relative to that of an average patient who does not receive the combination therapy described herein. As described herein, “progression-free survival” refers to the length of time during and after treatment of a cancer, that a selected subject lives with the disease, but does not get worse. For example, in some embodiments, progression-free survival is improved by at least ˜3 (e.g., at least ˜3, at least ˜4, at least ˜5, at least ˜6, at least ˜7, at least ˜8, at least ˜9, at least ˜10, at least ˜11, at least ˜12, ˜3, ˜4, ˜5, ˜6, ˜7, ˜8, ˜9, ˜10, ˜11, ˜12, or more) months. In some embodiments, progression-free survival is improved within a range having a lower limit selected from ˜3 months, ˜4 months, ˜5 months, ˜6 months, ˜7 months, ˜8 months, ˜9 months, ˜10 months, and ˜11 months, and an upper limit selected from ˜4 months, ˜5 months, ˜6 months, ˜7 months, ˜8 months, ˜9 months, ˜10 months, ˜11 months, and ˜12 months, in any combination thereof.

Suitable therapeutic benchmarks for treating a gastrointestinal tumor include, for example, inhibiting growth of the tumor, decreasing the size of the tumor, inhibiting proliferation of the tumor, and/or inhibiting metastasis of the tumor.

The effectiveness of the methods of the present application in treating the selected subject or treating the selected tumor may be evaluated, for example, by assessing changes in tumor burden and/or disease progression following treatment with the combination of therapeutic agents (e.g., the PARP inhibitor, oxaliplatin, and/or the antimetabolite) described herein according to the Response Evaluation Criteria in Solid Tumours (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). In some embodiments, tumor burden and/or disease progression is evaluated using imaging techniques including, e.g., X-ray, computed tomography (CT) scan, magnetic resonance imaging, mammography, and/or ultrasound (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). Tumor burden and/or disease progression may be monitored prior to, during, and/or following treatment with one or more of the therapeutic agents described herein.

In specific embodiments, the response to treatment with the methods described herein results in at least ˜1% (e.g., at least about 1%, at least ˜2%, at least ˜3%, at least ˜4%, at least ˜5%, at least ˜6%, at least ˜7%, at least ˜8%, at least ˜9%, at least ˜10%, at least ˜20%, at least ˜30%, at least ˜40%, at least ˜50%, at least ˜60%, at least ˜70%, at least ˜80%, at least ˜90%, at least ˜95%, at least ˜99%, ˜1%, ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, ˜99%, ˜˜100%) decrease in tumor size as compared to baseline tumor size. In some embodiments, the response to treatment results in a decrease in tumor size within a range having a lower limit selected from ˜˜1%, ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, and ˜99%, and an upper limit selected from ˜2%, ˜3%, ˜4%, ˜5%, ˜6%, ˜7%, ˜8%, ˜9%, ˜˜10^{%}, ˜˜20%, ˜30%, ˜40%, ˜50%, ˜60%, ˜70%, ˜80%, ˜90%, ˜95%, ˜99%, and ˜100%, in any combination thereof. Thus, the response to treatment with any of the methods described herein may be partial (e.g., at least a 30% reduction in tumor size, as compared to baseline tumor size) or complete (elimination of the tumor and/or prevention of tumor metastasis).

In certain embodiments, the methods described herein reduce the rate of tumor metastasis, growth, or proliferation in the selected subject/of the selected tumor by at least about 1% (e.g., at least about 1%, at least ˜2%, at least ˜3%, at least ˜4%, at least ˜5%, at least ˜6%, at least ˜7%, at least ˜8%, at least ˜9%, at least ˜10%, at least ˜20%, at least ˜30%, at least ˜40%, at least ˜50%, at least ˜60%, at least ˜70%, at least ˜80%, at least ˜90%, at least ˜95%, at least ˜99%, ˜1%, ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, ˜99%, ˜100%). In some embodiments, the rate of tumor metastasis, growth, or proliferation is reduced within a range having a lower limit selected from ˜1%, ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, and ˜99%, and an upper limit selected from ˜2%, ˜3%, ˜4%, ˜5%, ˜6%, ˜7%, 8%, ˜9%, ˜10%, ˜20%, ˜30%, ˜˜40%, ˜50%, ˜60%, ˜70%, ˜80%, ˜90%, ˜95%, ˜99%, and ˜100%, in any combination thereof.

As will be apparent to the skilled artisan, the therapeutic agents may be administered using any suitable method. By way of example, suitable modes of administration include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intraarterially, intralesionally, or by application to mucous membranes.

Suitable modes of local administration of the therapeutic agents and/or combinations disclosed herein include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent and the cancer to be treated.

In some embodiments, administering to the selected subject or the selected tumor is carried out in one or more 14-day cycles. By way of example, administering to the selected subject or the selected tumor may be carried out in at least two 14-day cycles, at least three 14-day cycles, or at least four 14-day cycles.

In some embodiments, at least the first cycle involves: (i) administering the PARP inhibitor on Day 1 at a dose of 40-200 mg (e.g., 40 mg, 50 mg, 60 mg, 70 mg, 80 mg, 90 mg, 100 mg, 120 mg, 130 mg, 140 mg, 150 mg, 160 mg, 170 mg, 180 mg, 190 mg, 200 mg); (ii) administering the oxaliplatin on Day 1 at a dose of 50-85 mg/m² (e.g., 50 and (iii) administering the antimetabolite on Day 1 at a dose of 1,200-2,400 mg/m² (e.g., 1,200 mg/m², 1,300 mg/ml, 1,400 mg/m², 1,500 mg/m², 1,600 mg/m², 1,700 mg/m², 1,800 mg/m², 1,900 mg/m², 2,000 mg/m², 2,100 mg/m², 2,200 mg/m², 2,300 mg/m², 2,400 mg/m²).

In some embodiments, each cycle involves (i) administering the PARP inhibitor on Day 1 at a dose of 40-200 mg (e.g., 40 mg, 50 mg, 60 mg, 70 mg, 80 mg, 90 mg, 100 mg, 120 mg, 130 mg, 140 mg, 150 mg, 160 mg, 170 mg, 180 mg, 190 mg, 200 mg). (ii) administering the oxaliplatin on Day 1 at a dose of 50-85 mg/m² (e.g., 50 mg/m², 55 mg/m², 60 mg/m², 65 mg/m², 70 mg/m², 75 mg/m², 80 mg/m², 85 mg/m²); and (iii) administering the antimetabolite on Day 1 at a dose of 1,200-2,400 mg/m² (e.g., 1,200 mg/m², 1,300 mg/m², 1,400 mg/m², 1,500 mg/m², 1,600 mg/m², 1,700 mg/m², 1,800 mg/m², 1,900 mg/m², 2,000 mg/m², 2,100 mg/m², 2,200 mg/m², 2,300 mg/m², 2,400 mg/m²).

In some embodiments, the PARP inhibitor is administered at a dose within a range having a lower limit selected from 40 mg, 50 mg, 60 mg, 70 mg, 80 mg, 90 mg, 100 mg, 120 mg, 130 mg, 140 mg. 150 mg, 160 mg, 170 mg, 180 mg, and 190 mg, and an upper limit selected from 50 mg, 60 mg, 70 mg, 80 mg, 90 mg, 100 mg, 120 mg, 130 mg, 140 mg, 150 mg, 160 mg, 170 mg, 180 mg, 190 mg, and 200 mg, in any combination thereof. In some embodiments, administering is carried out in at least two cycles, where the PARP inhibitor is administered at a lower dose in the second cycle than in the first cycle.

In some embodiments, oxaliplatin is administered at a dose within a range having a lower limit selected from 50 mg/m², 55 mg/m², 60 mg/m², 65 mg/m², 70 mg/m², 75 mg/m², and 80 mg/m², and an upper limit selected from 55 mg/m², 60 mg/m², 65 mg/m², 70 mg/m², 75 mg/m², 80 mg/m², and 85 mg/m², in any combination thereof.

In some embodiments, the antimetabolite is administered at a dose within a range having a lower limit selected from 1,200 mg/m², 1,300 mg/m², 1,400 mg/m², 1,500 mg/m², 1,600 mg/m². 1,700 mg/m², 1,800 mg/m², 1,900 mg/m², 2,000 mg/m², 2,100 mg/m², 2,200 mg/m², and 2,300 mg/m², and an upper limit selected from 1,300 mg/m², 1,400 mg/m², 1,500 mg/m², 1,600 mg/m², 1,700 mg/m², 1,800 mg/m², 1,900 mg/m², 2,000 mg/m², 2,100 mg/m², 2,200 mg/m². 2,300 mg/m², and 2,400 mg/m², in any combination thereof.

In some embodiments, at least the first cycle and/or at least one of the cycles and/or each cycle may further involve administering folinic acid on Day 1 at a dose of 1-400 mg/m² (e.g., 1 mg/m², 5 mg/m², 10 mg/m², 20 mg/m², 30 mg/m², 40 mg/m², 50 mg/m², 60 mg/m². 70 mg/m², 80 mg/m², 90 mg/m², 100 mg/m², 150 mg/m², 200 mg/m², 250 mg/m², 300 mg/m², 350 mg/m², 400 mg/m²). In some embodiments, the folinic acid is administered at a dose within a range having a lower limit selected from 1 mg/m², 5 mg/m², 10 mg/m², 20 mg/m², 30 mg/m², 40 mg/m², 50 mg/m², 60 mg/m², 70 mg/m², 80 mg/m², 90 mg/m², 100 mg/m², 150 mg/m², 200 mg/m², 250 mg/m², 300 mg/m², and 350 mg/m², and an upper limit selected from 5 mg/m², 10 mg/m², 20 mg/m², 30 mg/m², 40 mg/m², 50 mg/m², 60 mg/m², 70 mg/m², 80 mg/m², 90 mg/m², 100 mg/m², 150 mg/m², 200 mg/m², 250 mg/m², 300 mg/m², 350 mg/m², and 400 mg/m², in any combination thereof. In some embodiments, the folinic acid is leucovorin or levoleucovorin.

A further aspect of the technology described herein relates to a method of increasing sensitivity of a gastrointestinal tumor cell or gastrointestinal cancer cell to treatment with oxaliplatin. This method involves selecting a gastrointestinal tumor cell or gastrointestinal cancer cell, where the cell comprises a pathogenic mutation in one or more HR-DDR pathway genes and administering to the cell a PARP inhibitor in an amount effective to increase sensitivity of the cell to treatment with oxaliplatin and an antimetabolite.

The term “sensitivity” is a relative term which refers to an increase in the degree of effectiveness of a therapy (involving oxaliplatin and an antimetabolite) in reducing, inhibiting, and/or suppressing growth of gastrointestinal tumor cells or gastrointestinal cancer cells. The term “growth” as used herein, encompasses any aspect of the growth, proliferation, and progression of gastrointestinal tumor/cancer cells, including, e.g., cell division (i.e., mitosis), cell growth (e.g., increase in cell size), an increase in genetic material (e.g., prior to cell division), and metastasis. Reduction, inhibition, and/or suppression of cell growth includes, but is not limited to, inhibition of cell growth as compared to the growth of untreated or mock treated cells, inhibition of proliferation, inhibition of metastases, induction of cell senescence, induction of cell death, and reduction of cell size. An increase in sensitivity to a therapy may be measured by, e.g., using cell proliferation assays and/or cell cycle analysis assays.

In some embodiments, the sensitivity of the gastrointestinal tumor/cancer cells to treatment with oxaliplatin and an antimetabolite is increased by at least ˜1% (e.g., at least about 1%, at least ˜2%, at least ˜3%, at least ˜4%, at least ˜5%, at least ˜6%, at least ˜7%, at least ˜8%, at least ˜9%, at least ˜10%, at least ˜20%, at least ˜30%, at least ˜40%, at least 50%, at least ˜60%, at least ˜70%, at least ˜80%, at least ˜90%, at least ˜95%, at least ˜99%, ˜1%, ˜2%, 3%, ˜4%, ˜0.5%, ˜60%, ˜7%, ˜8%, ˜9%, ˜10%, ˜20%, ˜30%, ˜40%, ˜50%, ˜60%, ˜70%, ˜80%, ˜90° %, ˜95%, ˜99%, ˜100%) as compared to when the PARP inhibitor is not administered to the selected gastrointestinal tumor/cancer cell. In some embodiments, the sensitivity is increased within a range having a lower limit selected from ˜1%, ˜2%, ˜3%, ˜4%, ˜5%, ˜0.6%, ˜7%, ˜8%, ˜9%, ˜10%, ˜20%, ˜30%, ˜40%, ˜50%, ˜60%, ˜70%, ˜80%, ˜90%, ˜95%, and ˜99%, and an upper limit selected from ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, ˜99%, and ˜100%, in any combination thereof.

In some embodiments, the method of sensitizing the cell further involves administering to the cell the oxaliplatin and the antimetabolite together with or after administering the PARP inhibitor.

The method of increasing the sensitivity of a gastrointestinal tumor cell or gastrointestinal cancer cell to treatment with oxaliplatin described herein may be carried out in vitro, in vivo, or ex vivo. When methods described herein are carried out in vivo, selecting gastrointestinal tumor/cancer cell may involve selecting a subject/tumor/cancer as described herein and administering the PARP inhibitor as described herein to the selected subject/tumor/cancer.

In all aspects of the present technology that involve administering combination(s) of therapeutic agents (e.g., the PARP inhibitor, oxaliplatin, and/or the anti-metabolite described herein), the therapeutic agents may be administered before, during, or after the administration of any, some, or all of the other therapeutic agents described herein. In some embodiments, the PARP inhibitor, oxaliplatin, and/or the anti-metabolite are administered simultaneously. In other embodiments, the PARP inhibitor, oxaliplatin, and/or the anti-metabolite are administered sequentially.

In some embodiments, the therapeutic agents described herein are administered on the same day, about 24 hours apart, about 23 hours apart, about 22 hours apart, about 21 hours apart, about 20 hours apart, about 19 hours apart, about 18 hours apart, about 17 hours apart, about 16 hours apart, about 15 hours apart, about 14 hours apart, about 13 hours apart, about 12 hours apart, about 11 hours apart, about 10 hours apart, about 9 hours apart, about 8 hours apart, about 7 hours apart, about 6 hours apart, about 5 hours apart, about 4 hours apart, about 3 hours apart, about 2 hours apart, about 1 hour apart, about 55 minutes apart, about 50 minutes apart, about 45 minutes apart, about 40 minutes apart, about 35 minutes apart, about 30 minutes apart, about 25 minutes apart, about 20 minutes apart, about 15 minutes apart, about 10 minutes apart, or about 5 minutes apart. In some embodiments, the therapeutic agents described herein are administered about 1 day apart, about 2 days apart, about 3 days apart, about 4 days apart, about 5 days apart, about 6 days apart, or about 1 week apart.

In certain embodiments, the therapeutic agents described herein may be administered as part of a single formulation. Included are kits in which a PARP inhibitor, oxaliplatin, and an antimetabolite are contained together, for example as a copackaging arrangement, with instructions to administer them to the selected subject population described herein.

In carrying out the methods of the present application, administering may further involve administering folinic acid to the subject, tumor, or cell. In some embodiments, the folinic acid is leucovorin or levoleucovorin.

The therapeutic agents and combinations for use in the methods described herein can be formulated according to any available conventional method. Examples of preferred dosage forms include a tablet, a powder, a subtle granule, a granule, a coated tablet, a capsule, a syrup, a troche, an inhalant, a suppository, an injectable, an ointment, an ophthalmic ointment, an eye drop, a nasal drop, an ear drop, a cataplasm, a lotion and the like. In the formulation, generally used additives such as a diluent, a binder, an disintegrant, a lubricant, a colorant, a flavoring agent, and if necessary, a stabilizer, an emulsifier, an absorption enhancer, a surfactant, a pH adjuster, an antiseptic, an antioxidant and the like can be used. In addition, the formulation is also carried out by combining compositions that arc generally used as a raw material for pharmaceutical formulation, according to conventional methods. Examples of these compositions include, for example, (1) an oil such as a soybean oil, a beef tallow and synthetic glyceride; (2) hydrocarbon such as liquid paraffin, squalane and solid paraffin; (3) ester oil such as octyldodecyl myristic acid and isopropyl myristic acid; (4) higher alcohol such as cetostearyl alcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, a solid polyoxyethylene castor oil and polyoxyethylene polyoxypropylene block co-polymer; (8) water soluble macromolecule such as hydroxyethyl cellulose, polyacrylic acid, carboxyvinyl polymer, polyethyleneglycol, polyvinylpyrrolidone and methylcellulose; (9) lower alcohol such as ethanol and isopropanol; (10) multivalent alcohol such as glycerin, propyleneglycol, dipropyleneglycol and sorbitol; (11) a sugar such as glucose and cane sugar; (12) an inorganic powder such as anhydrous silicic acid, aluminum magnesium silicicate and aluminum silicate; (13) purified water, and the like.

Additives for use in the above formulations may include, for example, (1) lactose, corn starch, sucrose, glucose, mannitol, sorbitol, crystalline cellulose and silicon dioxide as the diluent; (2) polyvinyl alcohol, polyvinyl ether, methyl cellulose, ethyl cellulose, gum arabic, tragacanth, gelatine, shellac, hydroxypropyl cellulose, hydroxypropylmethyl cellulose, polyvinylpyrrolidone, polypropylene glycol-poly oxyethylene-block co-polymer, meglumine, calcium citrate, dextrin, pectin and the like as the binder; (3) starch, agar, gelatine powder, crystalline cellulose, calcium carbonate, sodium bicarbonate, calcium citrate, dextrin, pectic, carboxymethylcellulose/calcium and the like as the disintegrant; (4) magnesium stearate, talc, polyethyleneglycol, silica, condensed plant oil and the like as the lubricant; (5) any colorants whose addition is pharmaceutically acceptable is adequate as the colorant; (6) cocoa powder, menthol, aromatizer, peppermint oil, cinnamon powder as the flavoring agent; (7) antioxidants whose addition is pharmaceutically accepted such as ascorbic acid or alpha-tophenol.

The therapeutic agents and combinations for use in the methods described herein can be formulated into a pharmaceutical composition as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods described herein, and mixtures comprising two or more of such compounds, pharmaceutically acceptable salts of the compounds and pharmaceutically acceptable solvates of the compounds. Such compositions arc prepared in accordance with acceptable pharmaceutical procedures such as described in Remington: The Science and Practice of Pharmacy, 20th edition, ed. Alfonso R. Gennaro (2000), which is hereby incorporated by reference in its entirety.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

Reference to therapeutic agents described herein includes any analog, derivative, isomer, metabolite, pharmaceutically acceptable salt, pharmaceutical product, hydrate, N-oxide, crystal, polymorph, prodrug or any combination thereof.

The therapeutic agents in a free form can be converted into a salt, if need be, by conventional methods. The term “salt” used herein is not limited as long as the salt is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred as “pharmacologically acceptable salt” of the compounds disclosed herein.

In certain embodiments, the therapeutic agents disclosed herein may be in a prodrug form, meaning that it must undergo some alteration (e.g., oxidation or hydrolysis) to achieve its active form. For example, capecitabine is an oral 5-FU pro-drug that is converted to 5-FU by liver and tumor cells.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

The present technology may be further illustrated by reference to the following examples.

### EXAMPLES

The following examples are provided to illustrate embodiments of the present technology but are by no means intended to limit its scope.

### Example 1—Materials and Methods

### Patients

Patients with metastatic pancreatic adenocarcinoma with measurable disease (as per RECIST 1.1 (Eisenhauer et al., “New Response Evaluation Criteria in Solid Tumours: Revised RECIST Guideline (Version 1.1),” Eur. J Cancer 45:228-247 (2009), which is hereby incorporated by reference in its entirety)) were eligible. Patients were aged ≥18 years, had an Eastern Cooperative Oncology Group performance status score of ≤2, and had adequate organ and bone marrow function (hemoglobin ≥9.5 g/dL, absolute neutrophil count ≤1.5×10⁹/L, platelet count ≥75×10⁹/L, serum creatinine level <1.5 mg/dL, bilirubin level ≤2.5× upper limit of normal (ULN), and ALT/AST levels ≤3×ULN). For the Phase I portion of the study, patients were not selected based on prior therapy, family history, nor germline or tumor HR-DDR mutational status.

For the Phase II portion, there were two cohorts. Patients in the previously untreated cohort (Cohort A) did not have any prior systemic therapy for metastatic pancreatic cancer, though prior adjuvant chemotherapy was allowed if completed >6 months prior, and prior palliative radiation to the primary mass was allowable. For patients in the previously treated cohort (Cohort B), patients may have had any number of prior therapies, except for prior PARP inhibitor-based therapy. Also, for the Phase II portion of the study (both cohorts), patients who either had a known pathogenic germline or somatic mutation in one of the HR-DDR genes (e.g., BRCA1/2, PALB2, ATM) were preselected; and/or patients were eligible if they had a family history suggestive of a breast or ovarian cancer syndrome, as detailed in the NCCN guidelines (NCCN Clinical Practice Guidelines in Oncology, “Genetic/Familial High-Risk Assessment: Breast and Ovarian, Version 3.2019,” J. Natl. Compr. Canc. Netw. (2019), which is hereby incorporated by reference in its entirety), and summarized as one or more of the following:
(1) Personal history of breast cancer and one or more of the following:
   (a) Diagnosed ≤45 years old
   (b) Diagnosed at any age with one or more 1^{st}, 2^{nd}, or 3^{rd} degree relatives with breast cancer ≤50 years old and/or one or more 1^{st}, 2^{nd}, or 3^{rd} degree relatives with epithelial ovarian cancer at any age
   (c) Two primary breast cancers with the first diagnosed at ≤50 years old
   (d) Diagnosed ≤60 years old with a triple negative breast cancer
   (e) Diagnosed at any age with two or more 1^{st}, 2^{nd}, or 3^{rd} degree relatives with breast cancer at any age
   (f) Diagnosed at any age with two or more 1^{st}, 2^{nd}, or 3^{rd} degree relatives with pancreatic cancer or aggressive prostate cancer (Gleason score ≥7) at any age
   (g) 1^{st}, 2^{nd}, or 3^{rd} degree male relative with breast cancer
   (h) Ashkenazi Jewish descent
(2) Personal history of epithelial ovarian cancer
(3) Personal history of male breast cancer
(4) Personal history of pancreatic cancer and two or more 1^{st}, 2^{nd}, or 3^{rd} degree relatives with breast, epithelial ovarian, pancreatic, or aggressive prostate cancer (Gleason score ≥7) at any age

### Study Design and Treatment Schedule

The study was designed as a single center, Phase I/II, open label study of veliparib plus FOLFOX. At the time of study initiation, the dose and schedule for modified FOLFOX (de Gramont et al., “Leucovorin and Fluorouracil With or Without Oxaliplatin as First-Line Treatment in Advanced Colorectal Cancer,” J. Clin. Oncol. 18:2938-2947 (2000), which is hereby incorporated by reference in its entirety) was used (5-fluorouracil bolus 400 mg/m² Day 1; leucovorin 400 mg/m² Day 1; oxaliplatin 85 mg/m² Day 1; and 5-fluorouracil 2400 mg/m² continuous infusion over 46 hours, Days 1-3). Each cycle was 14 days. However, after the enrollment of the first 6 patients at the lowest dose of veliparib at which dose patients demonstrated prolonged (although not profound) myelosuppression, the 5-fluorouracil bolus was dropped from the FOLFOX regimen for all subsequent patients. With regards to the veliparib, for the Phase I portion, the dose of veliparib was escalated in a standard 3+3 design from the lowest dose of 40 mg twice a day (BID) days 1-7 of each 14-day cycle. Dose levels were 40 mg, 60 mg, 80 mg, 100 mg, 150 mg, 200 mg, and 250 mg. For the Phase II portion, the recommended Phase II dose (RP2D) was 200 mg of veliparib, though the protocol did allow for stepwise de-escalation to 150 mg and then to 100 mg for toxicity after the first cycle. Safety assessments were performed every 2 weeks for the first 4 cycles, then every 4 weeks thereafter. Tumor response was assessed radiographically every 8-12 weeks using Response Evaluation Criteria in Solid Tumors criteria version 1.1 (Eisenhauer et al., “New Response Evaluation Criteria in Solid Tumours: Revised RECIST Guideline (Version 1.1),” Eur. J. Cancer 45:228-247 (2009), which is hereby incorporated by reference in its entirety). Study treatment was continued without interruption in the absence of unacceptable toxicity or progressive disease (PD). The primary objective of the Phase I portion was to determine the recommended Phase II dose, with the primary endpoint being adverse events, as measured by the National Cancer Institute Common Terminology Criteria for Adverse Events, Version 4.03, which is hereby incorporated by reference in its entirety. For the Phase II portion, the primary endpoint was the objective response rate (ORR); key secondary endpoints included the disease control rate (DCR), defined as the percent of patients with complete or partial response, or stable disease after 4 cycles; progression-free survival (PFS); and overall survival (OS).

### Correlative Markers of Response to Therapy

For the Phase I portion of the study, plasma samples were obtained from patients on Day 1 (pre-dose), Day 3, and Day 7 for pharmacokinetic (PK) assessment of veliparib. Results were compared to historical controls to identify any effect on veliparib pharmacokinetics by oxaliplatin or 5-fluorouracil. In addition, all patients were mandated to undergo a pre-treatment tumor biopsy, and archived tumor samples were obtained as well. Next generation sequencing (NGS) of cancer-related genes was performed on tumor samples. Results from patients were captured, if ordered by the treating physician prior to enrollment, and sequencing was performed commercially by Foundation Medicine (FM), Caris Life Sciences, or through commercial germline testing labs such as Myriad or Invitae. For the patients who did not have commercial testing, NGS testing of samples was performed on a research basis by Tempus, Inc. The FM, Caris, and Tempus testing included similar panels, particularly for the HR-DDR genes. Patients were defined as harboring HR-DDR mutations if a known pathogenic mutation in one of the HR-DDR genes (including but not limited to: ARID1A, ATM, ATRX, MRE11A, NBN, PTEN, RAD50/51/51B, BARD1, BLM, BRCA1, BRCA2, BRIP1, FANCA/C/D2/E/F/G/L, PALB2, WRN, CHEK2, CHEK1, BAP1, FAM175A, SLX4, MLL2, or XRCC) was identified in a blood sample (germline) or tumor sample (somatic).

### Statistical Analysis

The primary objective of the Phase I portion of the trial was to determine the recommended Phase II dose, by assessing the safety and tolerability as determined by adverse events, defined by the National Cancer Institute Common Terminology Criteria for Adverse Events, Version 4.03, which is hereby incorporated by reference in its entirety. The efficacy assessments included the objective response rate, progression-free survival, and overall survival. For the Phase II portion, each cohort was designed to follow a Simon's two-stage optimal design (Simon R., “Optimal Two-Stage Designs for Phase II Clinical Trials,” Control Clin. Trials 10:1-10 (1989), which is hereby incorporated by reference in its entirety). For each cohort, 9 patients were accrued in the first Stage, and if at least 1 patient demonstrated an objective response, up to 24 patients were to be accrued in the second Stage. If 3 or more patients in the second stage demonstrated an objective response, then the treatment was to be considered to be sufficiently promising to warrant further testing. At the time this protocol was designed, the response rate for first-line standard of care gemcitabine was only 7% (Burris et al., “Improvements in Survival and Clinical Benefit With Gemcitabine as First-Line Therapy for Patients With Advanced Pancreas Cancer: A Randomized Trial,” J. Clin. Oncol. 15:2403-13 (1997), which is hereby incorporated by reference in its entirety), and there was no standard second-line therapy. Thus, the sample sizes of 9 and 24 patients and the decision rules in Stages 1 and 2 respectively, were designed to differentiate a 5% objective response rate from a 25% objective response rate at a 1-sided 10% significance level and 90% power. Patient characteristics, medical features at study entry, and adverse events at least possibly related to study therapy were tabulated overall and by study cohort. Differences in objective response rate and disease control rate among subgroups were compared with chi-square tests, using exact calculations as needed for small sample sizes. Overall survival was defined as the number of months from enrollment until death or last contact. Patients who were alive at the time of analysis were censored at their last contact. Progression-free survival was defined as the number of months from enrollment to progression or death, whichever occurred first. Patients who were alive and progression-free at the time of analysis were censored at their last tumor assessment. Kaplan-Meier curves were presented for overall survival and progression-free survival. Analyses were performed in SAS 9.3 [SAS Institute Inc., Cary, N.C., USA.] and figures were created using STATA 12.1 [StatCorp LP, College Station, Tex., USA].

The trial opened in January 2011 and the Phase I portion accrued rapidly. However, when enrollment was restricted in the Phase II portion to those patients with a known HR-DDR mutation, or a family history of a breast or ovarian cancer syndrome, the accrual rate slowed considerably, and the trial was closed to accrual in 2019 due to slow accrual, although, as discussed below, despite closing early, the primary endpoint had been met in the two Phase II cohorts.

### Example 2—Patient Characteristics and Treatment Cohorts

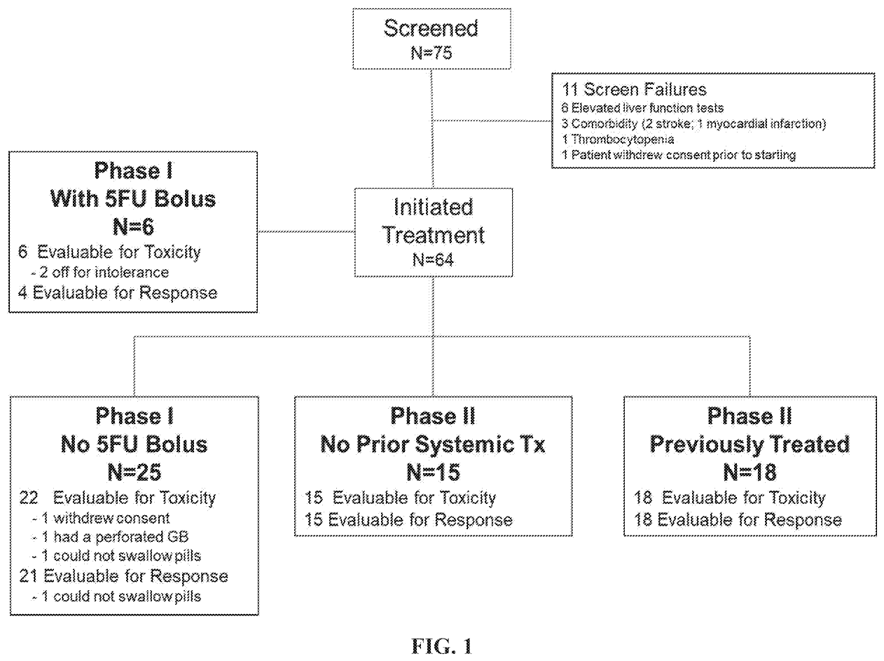

As shown in Table 1 below, 75 patients were consented and 64 patients initiated study treatment. FIG. 1 depicts the screen failure and enrollment into the different cohorts. 31 patients initiated study treatment in the Phase I portion; 15 patients who had received no prior systemic therapy for metastatic disease initiated study treatment in Cohort A of the Phase II portion: and 18 patients who were previously treated for metastatic disease initiated study treatment in Cohort B of the Phase II portion. Enrollment to both Phase II cohorts was stopped due to slow accrual. Patient characteristics are listed in Table 1. The median age for all 64 treated patients was 64 years (range 40-84 years); most patients had an Eastern Cooperative Oncology Group (ECOG) score of 0 or 1 (95%), and 56% of patients were male. In the Phase I, and previously treated cohort of the Phase II, patients had a median of 1 and 2 lines of prior therapy, respectively (range, 1-7).

TABLE 1
Patient Characteristics

| | | | | Phase II | |
|---|---|---|---|---|---|
| | | | Phase I | Untreated | Phase II Pre- |
| All Patients | | All N(%) | N (%) | N (%) | Treated N (%) |
| Category | Group | 64 (100%) | 31 (49%) | 15 (23%) | 18 (28%) |
| Age-median (min, | | 64 (40, 84) | 64 (46, 84) | 65 (40, 73) | 64 (52, 80) |
| max) | | | | | |
| Gender | Female | 28 (44%) | 21 (68%) | 10 (67%) | 8 (44%) |
| | Male | 36 (56%) | 10 (32%) | 5 (33%) | 10 (56%) |
| Race/Ethnicity | White/Non-Hispanic | 51 (80%) | 26 (84%) | 11 (73%) | 14 (78%) |
| | Black/Non-Hispanic | 10 (16%) | 4 (13%) | 3 (20%) | 3 (16%) |
| | Asian-Pl/Non-Hispanic | 2 (3%) | 0 (0%) | 1 (7%) | 1 (6%) |
| | Any/Hispanic | 1 (1%) | 1 (3%) | 0 (0%) | 0 (0%) |
| ECOG | 0 | 16 (25%) | 7 (23%) | 4 (27%) | 5 (28%) |
| | 1 | 45 (70%) | 23 (74%) | 9 (60%) | 13 (72%) |
| | 2 | 3 (5%) | 1 (3%) | 2 (13%) | 0 (0%) |
| Prior Platinum | Yes | 17 (27%) | 7 (23%) | 0 (0%) | 10 (56%) |
| | No | 47 (73%) | 24 (77%) | 15 (100%) | 8 (44%) |
| Family History | Yes | 44 (69%) | 12 (39%) | 15 (100%) | 17 (94%) |
| | No | 20 (31%) | 19 (61%) | 0 (0%) | 1 (6%) |
| Known HR-DDR | Yes | 19 (30%) | 2 (6%) | 5 (33%) | 12 (67%) |
| Mutation | No | 45 (70%) | 29 (94%) | 10 (67%) | 6 (33%) |

### Example 3—Phase I Portion

For the Phase I portion, veliparib was tested in dose ranges of 40 mg to 250 mg. Twenty-nine of the thirty-one patients were evaluable for dose limiting toxicities (DLT), with two patients withdrawing consent after one cycle (not for toxicity). In the 40 mg cohort. 3 out of 6 patients required significant (>2 weeks) treatment delays for Grade 2 or Grade 3 myelosuppression, primarily neutropenia and thrombocytopenia which did not rise to the level of a dose limiting toxicity, and the only protocol-defined dose limiting toxicity was a treatment delay of >3 weeks. Therefore, the protocol was amended to drop the 5-fluorouracil bolus. Thirty-one patients were enrolled in the Phase I, as depicted in FIG. 1. Only one other dose limiting toxicity occurred, which was in the 250 mg cohort. However, four of the six patients at 250 mg experienced significant Grade 3 or 4 myelosuppression. Therefore, 200 mg was selected as the recommended Phase II dose.

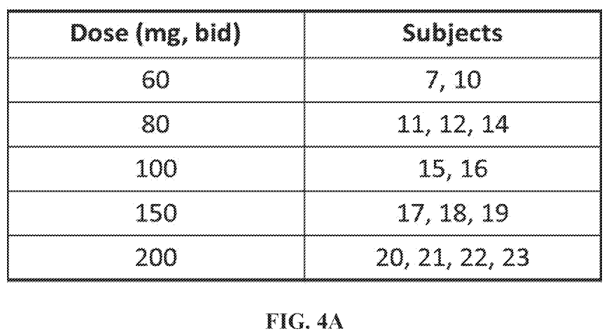

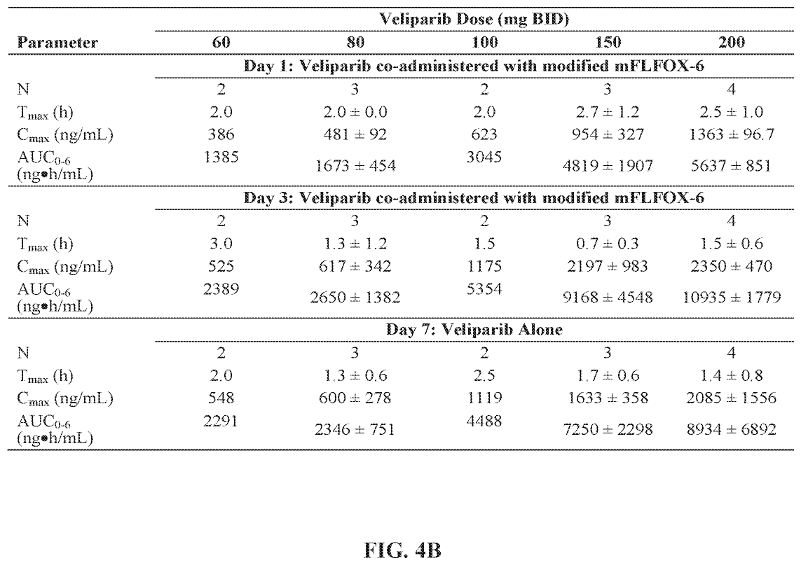

Serum samples were collected prior to treatment, on Day 3, and Day 7 for pharmacokinetic analysis. Pharmacokinetic analysis samples were available for 14 subjects in 5 dosing cohorts (FIGS. 4A-4B). The veliparib pharmacokinetic analysis data suggested that co-administration of FOLFOX had no apparent impact on veliparib pharmacokinetics.

### Example 4—Suspected Drug Related Adverse Events (all Cohorts)

All of the 64 patients who received treatment were evaluable for adverse events. Table 2 provides the number of patients experiencing adverse events by category and cohort that are possibly, probably, or definitely related to treatment. No grade 5 events occurred. Overall, the combination of veliparib and FOLFOX was well tolerated, although minor treatment adjustments were required over the course of therapy for most patients. Most patients experienced mild fatigue and nausea, but only 2% and 6%, respectively, were grade 3 or 4. Other notable Grade 3 or 4 non-hematologic adverse events included 1 patient each with a rash, diarrhea, and peripheral neuropathy. The primary toxicity of concern was myelosuppression, and 16% of patients experienced grade 3 or 4 neutropenia. One patient experienced grade 3 or 4 thrombocytopenia, and 2 patients had grade 3 or 4 anemia. Nearly all patients required a reduction in the dose of veliparib, 5-fluorouracil, and/or oxaliplatin due to myelosuppression or nausea prior to the first restaging imaging, and all patients who remained on study beyond 4 cycles required dose reduction. However, for the patients who remained on therapy beyond 4 cycles, most patients were well managed at a dose of veliparib of 150 mg, 5FU of 2400 mg/m², and oxaliplatin of 65 mg/m².

TABLE 2
Numbers (Percentages) of Patients Experiencing Adverse Events at Least Possibly
Related to Study Drug

| | | | | | | | | Phase II treated | | | Phase II untreated | | |
|---|---|---|---|---|---|---|---|---|---|---|---|---|---|
| | | All (N = 64) | | | Phase I N = 31 | | | N = 18 | | | N = 15 | | |
| AE | | Grade, N (%) | | | Grade, N (%) | | | Grade, N (%) | | | Grade, N (%) | | |
| Category | CTCAE Term | All | 3, 4 | 1, 2 | All | 3, 4 | 1, 2 | All | 3, 4 | 1, 2 | All | 3, 4 | 1, 2 |
| Hematologic | Neutropenia | 21(33) | 10(16) | 17(27) | 13(42) | 5(16) | 12(39) | 2(11) | 1(6) | 2(11) | 6(40) | 4(27) | 3(20) |
| | Thrombo- | 13(20) | 1(2) | 13(20) | 12(39) | 1(3) | 12(39 | | | | 1(7) | | 1(7) |
| | cytopenia | | | | | | | | | | | | |
| | Leukopenia | 11(17) | 3(5) | 10(16) | 9(29) | 3(10) | 8(26) | 1(6) | | 1(6) | 1(7) | | 1(7) |
| | Anemia | 6(9) | 2(3) | 4(6) | 4(13) | 1(3) | 3(10) | 1(6) | | 1(6) | 1(7) | 1(7) | |
| | Lymphopenia | 5(8) | 1(2) | 4(6) | 4(13) | 1(3) | 3(10) | | | | 1(7) | | 1(7) |
| Cardio-vascular | Hot Flashes | 1(2) | | 1(2) | 1(3) | | 1(3) | | | | | | |
| | Hypertension | 1(2) | | 1(2) | | | | 1(6) | | 1(6) | | | |
| | Hypotension | 1(2) | | 1(2) | | | | | | | 1(7) | | 1(7) |
| | Sinus | 1(2) | | 1(2) | 1(3) | | 1(3) | | | | | | |
| | tachycardia | | | | | | | | | | | | |
| Constitutional | Fatigue | 33(52) | 1(2) | 33(52) | 12(39) | | 12(39) | 11(61) | | 11(61) | 10(67) | 1(7) | 10(67) |
| | Anorexia | 14(27) | | 14(22) | 4(13) | | 4(13) | 6(33) | | 6(33) | 4(27) | | 4(27) |
| | Pain | 7(11) | | 7(11) | 5(16) | | 5(16) | 2(11) | | 2(11) | | | |
| | Allergic Reaction | 5(8) | | 5(8) | | | | 2(11) | | 2(11) | 3(20) | | 3(20) |
| | Weight Loss | 5(8) | | 5(8) | 2(6) | | 2(6) | 1(6) | | 1(6) | 2(13) | | 2(13) |
| | Fever | 2(3) | | 2(3) | 1(3) | | 1(3) | 1(6) | | 1(6) | | | |
| | Chills | 1(2) | | 1(2) | 1(3) | | 1(3) | | | | | | |
| | Febrile | 1(2) | | 1(2) | 1(3) | | 1(3) | | | | | | |
| | neutropenia | | | | | | | | | | | | |
| | Infusion related | 1(2) | | 1(2) | | | | 1(6) | | 1(6) | | | |
| | reaction | | | | | | | | | | | | |
| | Insomnia | 1(2) | | 1(2) | 1(3) | | 1(3) | | | | | | |
| | Malaise | 1(2) | | 1(2) | | | | | | | 1(7) | | 1(7) |
| Dermatologic | Rash maculo- | 2(3) | 1(2) | 2(3) | | | | 1(6) | 1(6) | 1(6) | 1(7) | | 1(7) |
| | papular | | | | | | | | | | | | |
| | Alopecia | 1(2) | | 1(2) | | | | | | | 1(7) | | 1 (7) |
| | Hand and foot | 1(2) | | 1(2) | | | | 1(6) | | 1(6) | | | |
| | syndrome | | | | | | | | | | | | |
| | Pruritus | 1(2) | | 1(2) | | | | | | | 1(7) | | 1(7) |
| Gastrointestinal | Nausea | 41(64) | 4(6) | 40(63) | 17(55) | | 17(55) | 13(72) | 2(11) | 13(72) | 11(73) | 2(13) | 10(67) |
| | Vomiting | 22(34) | 4(6) | 21(33) | 10(32) | | 10(32) | 6(33) | 2(11) | 6(33) | 6(40) | | 5(33) |
| | Diarrhea | 14(22) | 1(2) | 13(20) | 7(23) | 1(3) | 6(19) | 4(22) | | 4(22) | 3(20) | | 3(20) |
| | Constipation | 11(17) | | 11(17) | 5(16) | | 5(16) | 4(22) | | 4(22) | 2(13) | | 2(13) |
| | Mucositis oral | 7(11) | | 7(11) | 2(6) | | 2(6) | 3(17) | | 3(17) | 2(13) | | 2(13) |
| | Dysgeusia | 4(6) | | 4(6) | 1(3) | | 1(3) | 1(6) | | 1(6) | 2(13) | | 2(13) |
| | ALT increased | 3(5) | 1(2) | 3(5) | | | | 1(6) | 1(6) | | 2(13) | | 2(13) |
| | AST increased | 3(5) | 1(2) | 3(5) | | | | 1(6) | 1(6) | | 2(13) | | 2(13) |
| | Gastroesoph- | 3(5) | | 3(5) | 1(3) | | 1(3) | | | | 2(13) | | 2(13) |
| | ageal reflux | | | | | | | | | | | | |
| | disease | | | | | | | | | | | | |
| | Abdominal pain | 2(3) | | 2(3) | | | | 1(6) | | 1(6) | 1(7) | | 1(7) |
| | Bloating | 2(3) | | 2(3) | 2(6) | | 2(6) | | | | | | |
| | Dehydration | 2(3) | | 2(3) | 1(3) | | 1(3) | | | | 1(7) | | 1(7) |
| | Flatulence | 2(3) | | 2(3) | 1(3) | | 1(3) | | | | 1(7) | | 1(7) |
| | Gastroparesis | 2(3) | | 2(3) | 2(6) | | 2(6) | | | | | | |
| | Dry mouth | 1(2) | | 1(2) | 1(3) | | 1(3) | | | | | | |
| | Gastrointestinal | 1(2) | | 1(2) | 1(3) | | 1(3) | | | | | | |
| | pain | | | | | | | | | | | | |
| | Heartburn | 1(2) | | 1(2) | | | | | | | 1(7) | | 1(7) |
| | Hiccups | 1(2) | | 1(2) | | | | | | | 1(7) | | 1(7) |
| | Stomatitis | 1(2) | | 1(2) | | | | | | | 1(7) | | 1(7) |
| | Gastrointestinal | 3(5) | | 3(5) | 2(6) | | 2(6) | 1(6) | | 1(6) | | | |
| | disorders-Other | | | | | | | | | | | | |
| Genitourinary | Renal and | 1(2) | | 1(2) | 1(3) | | 1(3) | | | | | | |
| | urinary disorders- | | | | | | | | | | | | |
| | Other | | | | | | | | | | | | |
| | Vaginal | 1(2) | | 1(2) | | | | | | | 1(7) | | 1(7) |
| | discharge | | | | | | | | | | | | |
| Infection | Infections | 1(2) | | 1(2) | 1(3) | | 1(3) | | | | | | |
| | Leukocytosis | 1(2) | | 1(2) | 1(3) | | 1(3) | | | | | | |
| Musculoskeletal | Pain in extremity | 2(3) | | 2(3) | | | | 1(6) | | 1(6) | 1(7) | | 1(7) |
| | Edema limbs | 1(2) | | 1(2) | 1(3) | | 1(3) | | | | | | |
| | Muscle | 1(2) | | 1(2) | 1(3) | | 1(3) | | | | | | |
| | weakness-left | | | | | | | | | | | | |
| | sided | | | | | | | | | | | | |
| | Musculoskeletal- | 1(2) | | 1(2) | 1(3) | | 1(3) | | | | | | |
| | Other | | | | | | | | | | | | |
| | Joint range of | 1(2) | | 1(2) | | | | 1(6) | | 1(6) | | | |
| | motion decreased | | | | | | | | | | | | |
| Neurologic | Paresthesia | 17(27) | | 17(27) | 8(26) | | 8(26) | 6(33) | | 6(33) | 3(20) | | 3(20) |
| | Dysetgesua | 14(22) | | 14(22) | 8(26) | | 8(26) | 3(17) | | 3(17) | 3(20) | | 3(20) |
| | Headache | 5(8) | | 5(8) | 1(3) | | 1(3) | 3(17) | | 3(17) | 1(7) | | 1(7) |
| | Dizziness | 4(6) | | 4(6) | 1(3) | | 1(3) | 1(6) | | 1(6) | 2(13) | | 2(13) |
| | Cognitive | 2(3) | | 2(3) | 2(6) | | 2(6) | | | | | | |
| | disturbance | | | | | | | | | | | | |
| | Peripheral | 2(3) | 1(2) | 2(3) | | | | | | | 2(13) | 1(7) | 2(13) |
| | sensory | | | | | | | | | | | | |
| | neuropathy | | | | | | | | | | | | |
| | Vertigo | 2(3) | | 2(3) | 1(3) | | 1(3) | | | | 1(7) | | 1(7) |
| | Blurred vision | 1(2) | | 1(2) | | | | 1(6) | | 1(6) | | | |
| | Confusion | 1(2) | | 1(2) | 1(3) | | 1(3) | | | | | | |
| | Depression | 1(2) | | 1(2) | 1(3) | | 1(3) | | | | | | |
| | Eye disorders- | 1(2) | | 1(2) | 1(3) | | 1(3) | | | | | | |
| | Other | | | | | | | | | | | | |
| | Jaw Spasm | 1(2) | | 1(2) | | | | | | | 1(7) | | 1(7) |
| | Laryngopharyn- | 1(2) | | 1(2) | | | | 1(6) | | 1(6) | | | |
| | geal dysethesia | | | | | | | | | | | | |
| | Nervous system | 1(2) | | | | | | | | | 1(7) | 1(7) | |
| | disorders-Other | | | | | | | | | | | | |
| | Syncope | 1(2) | | | | | | | | | 1(7) | 1(7) | |
| | Tinnitus | 1(2) | | 1(2) | | | | | | | | | |
| Pulmonary | Dyspnea | 1(2) | | 1(2) | 1(3) | | 1(3) | 1(6) | | | | | |

### Example 5—Clinical Efficacy and Subgroup Assessment

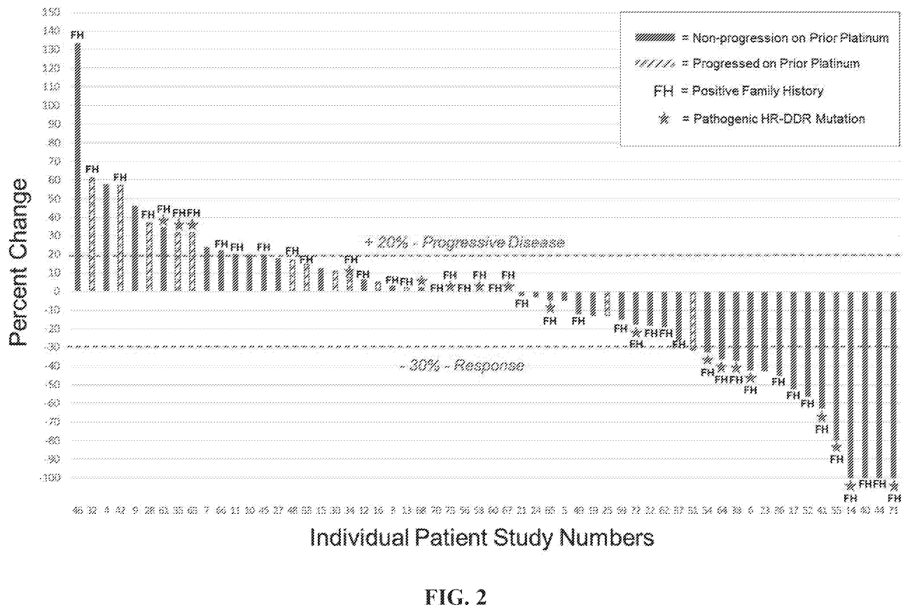

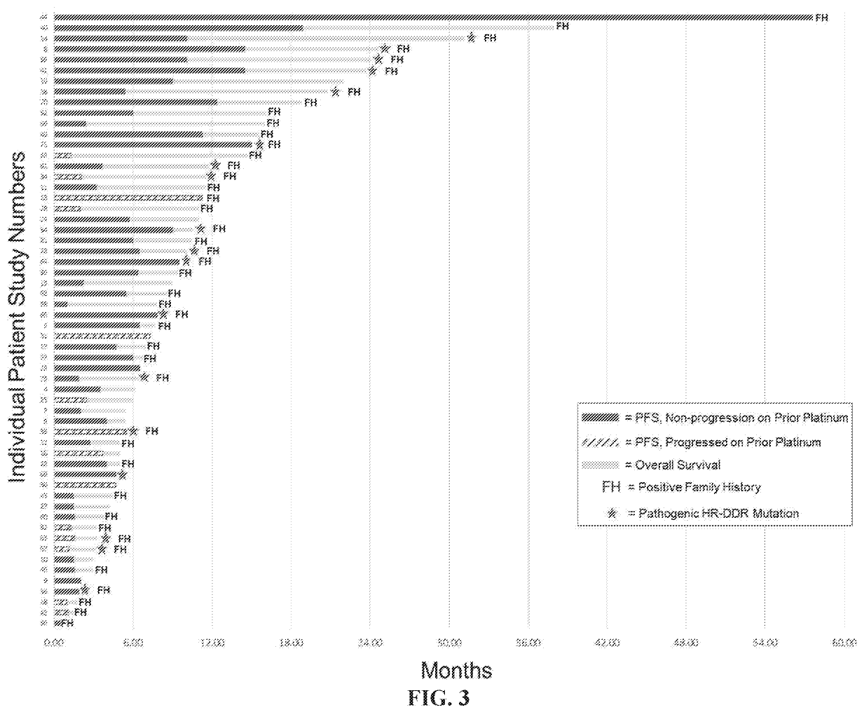

The primary endpoint was the objective response rate (ORR), and key secondary endpoints included the disease control rate (DCR) (stable disease (SD), partial response (PR), or complete response (CR) after 4 cycles); progression-free survival (PRS); and overall survival (OS). Of the 64 patients who received study treatment, 6 patients in the Phase I portion came off study prior to response evaluation, and thus were not evaluable for response. Table 3 presents the responses and survival times for the 58 evaluable patients. For the 58 response evaluable patients, the objective response rate was 26%, which included 11 partial responses and 4 complete responses. The waterfall plot in FIG. 2 demonstrates the responses for each patient. The disease control rate, progression-free survival, and overall survival were 52%, 4.0 months, and 7.8 months, respectively. The swimmers plot in FIG. 3 demonstrates the treatment duration for each patient. For the Phase I portion (n=25), patients were not pre-selected based on family history and known HR-DDR mutational status, and the objective response rate was 20%. For the two Phase II cohorts, combined (n=32), when patients were specifically selected based on family history (FH) or germline/somatic HR-DDR mutational status, the objective response rate increased to 31%. The objective response rate was 40% for patients who received no prior therapy (n=15), with a disease control rate, progression-free survival, and overall survival of 87%, 6.5 months, and 13.0 months, respectively. The objective response rate was 22% for the previously treated patients (N=18), with a disease control rate of 28%, 1.6 months, and 4.5 months, respectively.

TABLE 3
Patient Responses and Survival Times

| Subgroup (n) | | ORR (%) | DCR (%) | PFS (mos) | OS (mos) |
|---|---|---|---|---|---|
| All Response Evaluable Patients (58) | | 26 | 52 | 4.4 | 7.8 |
| Phase I Patients (25) | | 20 | 48 | 4.0 | 6.5 |
| Phase II Untreated (15) | | 40 | 87 | 6.5 | 15.0 |
| Phase II Previously Treated (18) | | 22 | 28 | 1.8 | 4.6 |
| Prior Platinums | Progression on Prior Platinum (14) | 7 | 14 | 2.1 | 5.3 |
| | No Prior Platinum (44) | 32 | 64 | 5.5 | 8.8 |
| Family History (FH) | (+) FH (43) | 30 | 53 | 5.4 | 9.5 |
| | No FH (15) | 13 | 47 | 3.8 | 5.5 |
| HR-DDR Mutations | HR-DDR mutated (18) | 44 | 61 | 6.0 | 10.4 |
| | Non-Mutated (40) | 18 | 48 | 3.6 | 6.9 |

| | | | | |
|---|---|---|---|---|
| HR-DDR Mutated, No progression on prior platinum (10) | 58 | 79 | 8.4 | 11.2 |

The efficacy in several patient subgroups was examined (Table 3). Without wishing to be bound by theory, the mechanisms of resistance to platinum-based therapies may overlap the mechanisms of resistance to PARP inhibitors. Correspondingly, the objective response rate for patients who received prior platinum-based therapy was only 7%, with a disease control rate, progression-free survival, and overall survival of 14%, 2.1 months, and 5.3 months, respectively (N=14). This is compared to an objective response rate of 32% for those who did not receive prior platinums, with a disease control rate, progression-free survival, and overall survival of 64%, 5.5 months, and 8.8 months, respectively (N=44).

Patients for the Phase II portion were selected for the presence of a family history suggestive of a breast or ovarian cancer syndrome, or a known mutation in one of the HR-DDR genes. 43 patients had a positive family history. The objective response rate for patients with a positive family history (irrespective of an HR-DDR mutation) was 30%, with a disease control rate, progression-free survival, and overall survival of 53%, 5.4 months, and 9.5 months, respectively (N=43). This is compared to an objective response rate of 13%, with a disease control rate, progression-free survival, and overall survival of 47%, 3.8 months, and 5.5 months, respectively for those with no such family history (N=15).

Finally, 23 out of 58 evaluable patients had NGS testing for germline or somatic pathogenic mutations in the HR-DDR genes. Of these, 14 patients had a BRCA1/2 mutation; 2 patients had an ATM mutation: and 1 patient had a PALB2 mutation, and 1 patient had a FANCG mutation. The objective response rate of the HR-DDR mutated patients was 44%, with a disease control rate of 53%, and a progression-free survival and overall survival of 5.4 months, and 9.5 months, respectively (N=18). The highest response rate was observed in HR-DDR mutated patients who had not previously received platinum-based therapy at 57% with a disease control rate of 79% which, surprisingly, was irrespective of the line of therapy (N=14).

### Discussion of Examples 1-5

Patients with metastatic pancreatic cancer are in desperate need of additional therapies. The modern chemotherapy regimens of FOLFIRINOX and gemcitabine+nab-paclitaxel have improved outcomes, but response rates are only 310% and 23%, respectively, and median overall survival remains <1 year (Conroy et al., “FOLFIRINOX Versus Gemcitabine for Metastatic Pancreatic Cancer,” N. Engl. J. Med. 364:1817-1825 (2011); Von Hoff et al., “Increased Survival in Pancreatic Cancer with Nab-Paclitaxel Plus Gemcitabine,” N. Engl. J. Med. 369:1691-1703 (2013), each of which is hereby incorporated by reference in its entirety). However, large scale sequencing efforts have revealed that a significant portion of pancreatic cancers harbor mutations in the HR-DDR genes, most commonly BRCA1L2 and ATM. Emerging data has revealed that patients with HR-DDR mutated metastatic pancreatic cancer can respond to PARP inhibitors, and even complete responses can be achieved (Domchek et al., “Efficacy and Safety of Olaparib Monotherapy in Germline BRCA1/2 Mutation Carriers with Advanced Ovarian Cancer and Three or More Lines of Prior Therapy.” Gynecol. Oncol. 140:199-203 (2016); Lowery et al., “An Emerging Entity: Pancreatic Adenocarcinoma Associated with a Known BRCA Mutation: Clinical Descriptors, Treatment Implications, and Future Directions,” Oncologist 16:1397-1402 (2011); O'Reilly et al., “Phase I Trial Evaluating Cisplatin, Gemcitabine, and Veliparib in 2 Patient Cohorts: Germline BRCA Mutation Carriers and Wild-Type BRCA Pancreatic Ductal Adenocarcinomam” Cancer (2018); O'Reilly et al., “Phase IB Trial of Cisplatin (C), Gemcitabine (G), and Veliparib (V) in Patients With Known or Potential BRCA or PALB2-Mutated Pancreas Adenocarcinoma (PC)” JCO 32:5s, (suppl: abstr 4023) (2014); Shroff et al., “Rucaparib Monotherapy in Patients with Pancreatic Cancer and a Known Deleterious BRCA Mutation,” JCO Precis. Oncol. (2018); Golan et al., “Overall Survival and Clinical Characteristics of Pancreatic Cancer in BRCA Mutation Carriers,” Br. J. Cancer 111:1132-1138 (2014), each of which is hereby incorporated by reference in its entirety). However, the activity of single agent PARP inhibitors has been limited. For example, out of 24 patients with BRCA1/2-mutated tumors, only 4 responded to therapy with olaparib (Domchek et al., “Efficacy and Safety of Olaparib Monotherapy in Germline BRCA1/2 Mutation Carriers with Advanced Ovarian Cancer and Three or More Lines of Prior Therapy,” Gynecol. Oncol. 140:199-203 (2016), which is hereby incorporated by reference in its entirety). Similarly, out of 19 patients with BRCA1/2-mutated pancreatic cancer, there were 4 responses to rucaparib (Shroff et al., “Rucaparib Monotherapy in Patients with Pancreatic Cancer and a Known Deleterious BRCA Mutation,” JCO Precis. Oncol. (2018), which is hereby incorporated by reference in its entirety). Shroff et al., “Rucaparib Monotherapy in Patients with Pancreatic Cancer and a Known Deleterious BRCA Mutation,” JCO Precis. Oncol. (2018), which is hereby incorporated by reference in its entirety, did present more detailed data on the prior platinum exposure for patients treated with rucaparib, and similar to the results presented herein, the majority of the responders were in patients who had either not been exposed to, or had not progressed on prior platinum-based therapy.

The Phase I/II trial of FOLFOX+veliparib results set forth in Examples 1-5 herein demonstrate that patients with metastatic pancreatic cancer can respond to this combination (objective response rate=21%). For the two Phase II cohorts, while the study was closed early due to slow accrual, the protocol-defined primary endpoint in both cohorts was met, with at least 3 responders in each cohort. In fact, 40% of untreated, and 18% of previously treated patients achieved a partial response or complete response. When patients were selected based on prior family history and/or the presence of HR-DDR mutations, the response rate increased to 45%, and in fact that highest responses were observed in patients whose tumors harbored HR-DDR mutations, and who had not been exposed to prior platinum-based therapies (objective response rate=60%). The combination was well tolerated, and, for the patients with long-term (>4 month) control of disease, a strategy of “maintenance” therapy without the oxaliplatin was able to maintain disease control for a prolonged period, as demonstrated by the swimmer's plot (FIG. 3).

It is possible that patients with HR-DDR mutations would respond to platinum-based therapy alone. However, in the results presented herein, two patients whose disease did not previously respond to FOLFIRINOX (i.e., stable disease only), did have a response to FOLFOX+veliparib. Similarly there were two apparent patients who did not respond to platinum, but did respond to rucaparib in Shroff et al., “Rucaparib Monotherapy in Patients with Pancreatic Cancer and a Known Deleterious BRCA Mutation,” JCO Precis. Oncol. (2018), which is hereby incorporated by reference in its entirety.

There is an ongoing debate on the functional role of PARP inhibition in the treatment of HR-DDR mutated cancers. PARP inhibitors can have multiple effects in mediating DNA damage, leading to cancer cell death. Originally, PARP inhibitors were demonstrated to inhibit the catalytic activity of the PARP-1 enzyme, thus inhibiting single strand repair, particularly after co-treatment with a DNA-damaging chemotherapy. This mechanism was the foundation for the synergy demonstrated for the combination of veliparib and various chemotherapies. However, a second critical role of some PARP inhibitors involves the trapping of the PARP enzyme at the site of DNA damage. The trapped PARP enzyme complex results in replication fork arrest, leading ultimately to mitotic catastrophe and apoptotic cell death. Several PARP inhibitors such as olaparib, niraparib, rucaparib, and talozaparib can achieve PARP trapping and replication fork arrest, and thus are active as single agents. Veliparib can only achieve catalytic inhibition of the PARP enzyme, and thus, appears to be most effective in combination with DNA damaging agents. Without wishing to be bound by theory, while veliparib may not be effective as a single agent, the tradeoff may be that the limited spectrum of activity of veliparib may also allow for the safe combination with DNA damaging agents, such as radiation and chemotherapy. By contrast, many of the PARP trapping inhibitors have been too toxic to use in combination with DNA damaging chemotherapies (Chen et al., “A Phase I Study of Olaparib and Irinotecan in Patients with Colorectal Cancer: Canadian Cancer Trials Group IND 187,” Invest. New Drugs 34(4):450-457 (2016); Samol et al., “Safety and Tolerability of the Poly(ADP-ribose) Polymerase (PARP) Inhibitor. Olaparib (AZD2281) in Combination with Topotecan for the Treatment of Patients with Advanced Solid Tumors: A Phase I Study,” Invest. New Drugs 30(4):1496-1500 (2012): Balmaña et al., “Phase I Trial of Olaparib in Combination with Cisplatin for the Treatment of Patients with Advanced Breast, Ovarian and other Solid Tumors,” Ann. Oncol. 25(8):1656-1663 (2014); Rajan et al., “A Phase I Combination Study of Olaparib with Cisplatin and Gemcitabinc in Adults with Solid Tumors,” Clin. Cancer Res. 18(8):2344-2351 (2012): Dhawan et al., “Differential Toxicity in Patients with and without DNA Repair Mutations: Phase I Study of Carboplatin and Talazoparib in Advanced Solid Tumors,” Clin. Cancer Res. 23(21):6400-6410 (2017), each of which is hereby incorporate by reference in its entirety).

The results presented herein emphasize the need to identify patients whose disease harbors underlying HR-DDR mutations. The NCCN has recently recommended that all patients with pancreatic cancer undergo germline testing for the presence of an inherited predisposition to the development of pancreatic cancer. As demonstrated herein, using a combination therapy that includes a PARP inhibitor, in combination with oxaliplatin and an antimetabolite, can improve outcomes for pancreatic cancer patients identified as having underlying HR-DDR mutations and/or a family history suggestive of a breast or ovarian cancer syndrome. Furthermore, considering that oxaliplatin is commonly used to treat other gastrointestinal cancers, it is expected that this combination therapy can improve outcomes for such patients who have other gastrointestinal cancers.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of treating gastrointestinal cancer in a subject, said method comprising:
selecting a subject, wherein the subject (i) has been diagnosed with a gastrointestinal cancer and (ii) has (a) a pathogenic mutation in one or more homologous recombination-DNA damage repair (HR-DDR) pathway genes and/or (b) a family history suggestive of a breast or ovarian cancer syndrome; and
administering to the subject an effective amount of a Poly(ADP ribose) polymerase (PARP) inhibitor, in combination with oxaliplatin and an antimetabolite.

2. The method according to claim 1, wherein the subject has adequate organ and bone marrow function.

3. The method according to claim 1 or claim 2, wherein said amount is effective to halt disease progression in the subject, inhibit malignant tumor growth in the subject, inhibit metastasis of the cancer in the subject, and/or reduce tumor size in the subject.

4. A method of treating a gastrointestinal tumor in a subject, said method comprising:
selecting a gastrointestinal tumor of a subject, wherein the tumor has a pathogenic mutation in one or more homologous recombination-DNA damage repair (HR-DDR) pathway genes and/or the subject has a family history suggestive of a breast or ovarian cancer syndrome; and
administering to the tumor an effective amount of a Poly(ADP ribose) polymerase (PARP) inhibitor, in combination with oxaliplatin and an antimetabolite.

5. The method according to claim 4, wherein said amount is effective to inhibit growth of the tumor, decrease the size of the tumor, inhibit proliferation of the tumor, and/or inhibit metastasis of the tumor.

6. The method according to any one of claims 1-5, wherein the subject has a family history suggestive of a breast or ovarian cancer syndrome.

7. The method according to any one of claims 1-6, wherein the subject has received systemic treatment with a platinum based chemotherapy, for any disorder, prior to said selecting.

8. The method according to claim 7, wherein the disorder did not progress in the subject following said prior treatment.

9. The method according to any one of claims 1-6, wherein the subject has not received systemic treatment with a platinum based chemotherapy prior to said selecting.

10. The method according to any one of claims 1-9, wherein said administering is carried out in at least one 14-day cycle.

11. The method according to claim 10, wherein said administering is carried out in at least four 14-day cycles.

12. The method according to claim 10 or claim 11, wherein the first cycle comprises:
(i) administering the PARP inhibitor on Day 1 at a dose of 40-200 mg;
(ii) administering the oxaliplatin on Day 1 at a dose of 50-85 mg/m²;
(iii) administering the antimetabolite on Day 1 at a dose of 1,200-2,400 mg/m².

13. The method according to claim 12, wherein said first cycle further comprises:
administering folinic acid on Day 1 at a dose of 1-400 mg/m².

14. The method according to claim 10 or claim 11, wherein each cycle comprises:
(i) administering the PARP inhibitor on Day 1 at a dose of 40-200 mg;
(ii) administering the oxaliplatin on Day 1 at a dose of 50-85 mg/m²; and
(iii) administering the antimetabolite on Day 1 at a dose of 1,200-2,400 mg/m².

15. The method according to claim 14, wherein each cycle further comprises:
administering folinic acid on Day 1 at a dose of 1-400 mg/m².

16. The method according to any one of claim 13 or claim 15, wherein the folinic acid is leucovorin or levoleucovorin.

17. The method according to any one of claims 10-16, wherein said administrating is carried out in at least two cycles, wherein the PARP inhibitor is administered at a lower dose in the second cycle than in the first cycle.

18. A method of increasing sensitivity of a gastrointestinal tumor cell or gastrointestinal cancer cell to treatment with oxaliplatin, said method comprising:
selecting a gastrointestinal tumor cell or gastrointestinal cancer cell, wherein the cell comprises a pathogenic mutation in one or more homologous recombination-DNA damage repair (HR-DDR) pathway genes; and
administering to the cell a Poly(ADP ribose) polymerase (PARP) inhibitor in an amount effective to increase sensitivity of the cell to treatment with oxaliplatin and an antimetabolite.

19. The method according to claim 18, further comprising: administering to the cell the oxaliplatin and the antimetabolite together with or after said administering the PARP inhibitor.

20. The method according to claim 18 or claim 19, wherein the method is carried out in vitro.

21. The method according to claim 18 or claim 19, wherein the method is carried out in vivo.

22. The method according to any one of claims 1-17, wherein the subject or the tumor has a pathogenic mutation in one or more HR-DDR pathway genes.

23. The method according to any one of claims 18-22, wherein the one or more HR-DDR pathway genes is selected from the group consisting of ARID1A, ATM, ATRX, MRE11A, NBN, PTEN, RAD50/51/51B, BARD1, BLM, BRCA1, BRCA2, BRIP1, FANCA/C/D2/E/F/G/L, PALB2, WRN, CHEK2, CHEK1, BAP1, FAM175A, SLX4, MLL2, and XRCC.

24. The method according to claim 22 or claim 23, wherein the one or more HR-DDR pathway genes is BRCA1/2 or PALB2.

25. The method according to any one of claims 22-24, wherein the mutation is a germline mutation.

26. The method according to any one of claims 22-24, wherein the mutation is a somatic mutation.

27. The method according to any one of claims 1-26, wherein the PARP inhibitor is veliparib (ABT-888).

28. The method according to any one of claims 1-17 and 19-27, wherein the antimetabolite is 5-fluorouracil or S-1.

29. The method according to any one of claims 1-17 and 19-28, wherein the PARP inhibitor, the oxaliplatin, and the antimetabolite are administered simultaneously.

30. The method according to any one of claims 1-17 and 19-29, wherein the PARP inhibitor, the oxaliplatin, and the antimetabolite are administered sequentially.

31. The method according to any one of claims 1-12, 14, 17, and 19-30, wherein said administering further comprises: administering folinic acid to the subject, tumor, or cell.

32. The method according to claim 31, wherein the folinic acid is leucovorin or levoleucovorin.

33. The method according to any one of claims 1-32, wherein the subject is a mammalian subject or the cell is a mammalian cell.

34. The method according to claim 33, wherein the mammal is a human.

35. The method according to any one of claims 1-34, wherein the gastrointestinal cancer/tumor is selected from the group consisting of oral cavity cancer/tumor, pharyngeal cancer/tumor, esophageal cancer/tumor, gastric cancer/tumor, small intestinal cancer/tumor, cecal cancer/tumor, colon cancer/tumor, rectal cancer/tumor, anal cancer/tumor, salivary gland cancer/tumor, liver cancer/tumor, pancreatic cancer/tumor, biliary cancer/tumor (bile duct cancer/tumor), gall bladder cancer/tumor, and peritoneal cancer/tumor.

36. The method according to any one of claims 1-35, wherein the gastrointestinal cancer/tumor is a pancreatic cancer/tumor.

37. The method according to claim 36, wherein the pancreatic cancer/tumor is an exocrine cancer/tumor.

38. The method according to claim 36 or claim 37, wherein the cancer/tumor is selected from the group consisting of acinar cell carcinoma, adenocarcinoma (ductal adenocarcinoma), adenosquamous carcinoma, anaplastic carcinoma, cystadenocarcinoma, duct-cell carcinoma, giant-cell carcinoma (osteoclastoid type), a giant cell tumor, intraductal papillary-mucinous neoplasm (IPMN), mixed-cell carcinoma, mucinous (colloid) carcinoma, mucinous cystadenocarcinoma, papillary adenocarcinoma, pleomorphic giant-cell carcinoma, serous cystadenocarcinoma, small-cell (oat-cell) carcinoma, solid tumors, and pseudopapillary tumors.

39. The method according to any one of claims 1-35, wherein the gastrointestinal cancer/tumor is an oral cavity cancer/tumor, pharyngeal cancer/tumor, or oralpharyngeal cancer/tumor.

40. The method according to claim 39, wherein the cancer/tumor is selected from the group consisting of carcinoma in situ and verrucous carcinoma/tumor.

41. The method according to any one of claims 1-35, wherein the gastrointestinal cancer/tumor is an esophageal cancer/tumor.

42. The method according to claim 41, wherein the cancer/tumor is selected from the group consisting of adenocarcinoma, squamous cell carcinoma, small cell carcinoma, lymphoma, melanomas, and sarcoma.

43. The method according to any one of claims 1-35, wherein the gastrointestinal cancer/tumor is a gastric cancer/tumor.

44. The method according to claim 43, wherein the cancer/tumor is selected from the group consisting of adenocarcinoma (distal stomach cancer/tumor, proximal stomach cancer/tumor, diffuse stomach cancer/tumor), gastrointestinal stromal tumors, carcinoid tumors, lymphoma, squamous cell carcinoma, small cell carcinoma, leiomyosarcoma, signet ring cell carcinoma, gastric lymphoma (MALT lymphoma), and linitis plastica.

45. The method according to any one of claims 1-35, wherein the gastrointestinal cancer/tumor is a small intestinal cancer/tumor.

46. The method according to claim 45, wherein the cancer/tumor is selected from the group consisting of adenocarcinoma, carcinoid tumors, lymphomas, and sarcomas (gastrointestinal stromal tumors).

47. The method according to any one of claims 1-35, wherein the gastrointestinal cancer/tumor is a cecal cancer/tumor.

48. The method according to claim 47, wherein the cancer/tumor is selected from the group consisting of adenocarcinoma, squamous cell carcinoma, and sarcoma (leiomyosarcoma).

49. The method according to any one of claims 1-35, wherein the gastrointestinal cancer/tumor is a colon cancer/tumor, rectal cancer/tumor, or colorectal cancer/tumor.

50. The method according to claim 49, wherein the cancer/tumor is selected from the group consisting of adenocarcinoma, carcinoid tumors, gastrointestinal stromal tumors, lymphomas, and sarcomas.

51. The method according to any one of claims 1-35, wherein the gastrointestinal cancer/tumor is an anal cancer/tumor.

52. The method according to claim 51, wherein the cancer/tumor is selected from the group consisting of carcinoma in situ (Bowen disease), squamous cell carcinomas (e.g., cloacogenic carcinoma), adenocarcinomas, basal cell carcinomas, melanomas, and gastrointestinal stromal tumors.

53. The method according to any one of claims 1-35, wherein the gastrointestinal cancer/tumor is a salivary gland cancer/tumor.

54. The method according to claim 53, wherein the cancer/tumor is selected from the group consisting of adenoid cystic carcinoma, mucoepidermoid carcinoma, and polymorphous low-grade adenocarcinoma.

55. The method according to any one of claims 1-35, wherein the gastrointestinal cancer/tumor is a liver cancer/tumor.

56. The method according to claim 55, wherein the cancer/tumor is selected from the group consisting of hepatocellular carcinoma (e.g., fibrolamellar hepatocellular carcinoma), intrahepatic cholangiocarcinoma (bile duct cancer), angiosarcoma, hemangiosarcoma, and hepatoblastoma.

57. The method according to any one of claims 1-35, wherein the gastrointestinal cancer/tumor is a biliary cancer/tumor (bile duct cancer/tumor).

58. The method according to claim 57, wherein the cancer/tumor is selected from the group consisting of adenocarcinomas, sarcomas, lymphomas, and small cell cancers/tumors.

59. The method according to any one of claims 1-35, wherein the gastrointestinal cancer/tumor is a gall bladder cancer/tumor.

60. The method according to claim 59, wherein the cancer/tumor is selected from the group consisting of adenocarcinomas (papillary adenocarcinoma), adenosquamous carcinomas, squamous cell carcinomas, and carcinosarcomas.

61. The method according to any one of claims 1-35, wherein the gastrointestinal cancer/tumor is a peritoneal cancer/tumor.

62. The method according to claim 61, wherein the cancer/tumor is selected from the group consisting of peritoneal carcinoma, peritoneal mesothelioma, and desmoplastic small round cell tumor.

63. The method according to any one of claims 1-62, wherein the gastrointestinal cancer/tumor is a metastatic gastrointestinal cancer/tumor.
